(19) United States
(12) Patent Application Publication
(10) Pub. No.: US 2024/0350667 A1
(43) Pub. Date: Oct. 24, 2024

(54) Method of Enhancing Gene Therapy by Targeting cGAS-STING Pathway

(71) Applicant: Spark Therapeutics, Inc., Philadelphia, PA (US)

(72) Inventors: Xavier Anguel, Barcelona (ES); Pedro Cejas, Oreland, PA (US); Ali Nahvi, Needham, MA (US); Mustafa Yazicioglu, III, Broomall, PA (US); Rui Zhang, Winchester, MA (US)

(21) Appl. No.: 18/685,681

(22) PCT Filed: Jul. 22, 2022
(86) PCT No.: PCT/US2022/074084

§ 371 Date: Feb. 22, 2024

Related U.S. Application Data
(60) Provisional application No. 63/203,451, filed on Jul. 23, 2021.

(51) Int. Cl. A61K 48/00; A61K 9/127; A61K 9/51; A61K 31/404; A61K 31/437; A61K 45/06; C12N 15/88

(52) U.S. Cl. CPC A61K 48/005; A61K 9/127; A61K 9/5123; A61K 31/404; A61K 31/437; A61K 45/06; A61K 48/0083; C12N 15/88

(57) ABSTRACT

The present invention features method and composition that can be used to facilitate intracellular delivery of DNA to a subject. The provided methods and compositions employ a nanoparticle for intracellular DNA delivery and a cytosolic DNA-sensing inhibitor. The cytosolic DNA-sensing inhibitor is provided to decrease the subject's immune response that can be stimulated by the DNA.

### FIELD OF THE INVENTION

The invention relates to the field of intracellular non-viral DNA delivery to a subject. The provided methods and composition having different uses including gene therapy.

### REFERENCE TO SEQUENCE LISTING SUBMITTED ELECTRONICALLY

The contents of the electronic sequence listing (SequenceListing_9WO1.xml; Size: 3,810 bytes; and Date of Creation: Jul. 14, 2022) is herein incorporated by reference in its entirety.

### BACKGROUND OF THE INVENTION

Gene therapy involves using nucleic acid to modify a subject's DNA to achieve a beneficial effect. Gene modification can be performed using different strategies including gene augmentation, gene suppression and genome editing. (Anguela and High Annu. Rev. Med. 2019, 70, 73; and Li et al., Signal Transduction and Targeted Therapy 2020, 5, 1.)

An effective delivery system for nucleic acid is important for successful gene therapy. Successful delivery of the nucleic acid provides a sufficient amount to a target cell to achieve a beneficial effect without producing an unacceptable adverse reaction. The delivery system should protect the genetic material from enzymatic degradation, have a sufficiently long lifetime in the body, be able to reach the site within the body where it is needed, have tolerable toxicity, and be able to cross the cell membrane.

Gene therapy vectors can be broadly categorized as viral and non-viral. Each type of vector has advantages and disadvantages. Viral vectors are generally more efficient at delivering genetic material to a cell, but have a greater potential for immunogenicity, toxin production and insertional mutagenesis, and a more limited transgenic capacity size. Advantages of non-viral vectors include greater transgene capacity, the ability to dose subjects with pre-existing antibodies to vector capsid, and greater ability to re-dose a subject. Challenges associated with non-viral delivery can include lower transfection efficiency, potential nucleic acid degradation, innate immunity, low efficiency of gene delivery to somatic targets and lower in vivo gene expression levels than viral approaches. (Hardee et al., Genes 2017, 8, 65 and Nayerossadat et al., Adv. Biomed Res. 2012, 1, 27.)

### BRIEF SUMMARY OF THE INVENTION

The present invention features method and composition that can be used to facilitate intracellular delivery of DNA to a subject. The provided methods and compositions employ a nanoparticle for intracellular DNA delivery, and a cytosolic DNA-sensing inhibitor. The cytosolic DNA-sensing inhibitor is provided to decrease the subject's immune response that can be stimulated by the DNA.

Thus, a first aspect of the present invention describes a method of intracellular delivery of DNA comprising administering to a subject:
a) a cytosolic DNA-sensing inhibitor selected from the group consisting of a cyclic GMP-AMP synthase-stimulator of interferon genes (cGAS-STING) pathway inhibitor and an inflammasome pathway inhibitor; and
b) a nanoparticle comprising the DNA.

Another aspect of the present invention describes a nanoparticle comprising (a) a DNA and (b) a cytosolic DNA-sensing inhibitor selected from the group consisting of a cGAS-STING pathway inhibitor and an inflammasome pathway inhibitor.

Additional aspects of the present invention include pharmaceutical compositions containing the nanoparticles described herein, pharmaceutical compositions for uses described herein, and preparation of medicaments for uses described herein. Pharmaceutical compositions for uses described herein can provide a DNA vector comprising a transgene for use in a patient that is administered prior to, concomitantly with, of after, a cytosolic DNA-sensing inhibitor. Similarly, preparation of medicaments for uses described herein can involve preparation of a pharmaceutical composition containing a DNA vector comprising a transgene for use in a patient that prior to, concomitantly with, or after, is administered a cytosolic DNA-sensing inhibitor.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIG. 1 illustrates results from an in vitro study in cultured cells on the ability of small molecule STING inhibitor (H-151) to inhibit DNA-LNP-induced interferon regulatory factor (IRF) activation. Two amounts of H-151 were used (0.2 ng and 2.1 ng). The open bar at left indicates cells treated with DNA-LNP without any H-151 inhibitor (0). Where H-151 was added (0.2 and 2.1), open boxes indicate soluble H-151 (soluble), and shaded boxes indicate the H-151 was encapsulated in the DNA-LNP (LNP-encapsulated).

FIGS. 2A, 2B, 2C, and 2D illustrate results from an in vivo study on the ability of H-151 encapsulated into DNA-LNP to inhibit DNA-LNP-induced IFN-β (FIG. 2A), IFN-α (FIG. 2B), IFN-γ (FIG. 2C) and IL-6 (FIG. 2D).

FIGS. 3A, 3B, 3C, 3D, 3E, 3F, 3G, and 3H illustrate results from an in vivo study on the ability of RO3150, GSK690693, and dexamethasone to inhibit cytokine/chemokine levels induced by DNA-LNP.

FIGS. 4A, 4B, 4C, 4D, and 4E, illustrate the impact of STING signaling ablation on DNA-LNP tolerability and transgene expression in mice dosed with DNA-LNP gene therapy. The inflammatory response to DNA-LNP was assessed in wild type mice having a functional STING (WT) and mice harboring the Goldenticket missense mutation for the cytosolic double-stranded DNA (dsDNA) sensor (STING (Gt)). Baseline cytokine levels were determined from pooled plasma samples from WT mice having not received any DNA-LNP (Baseline). In mice dosed with DNA-LNP, STING (Gt) mice had lower plasma IL-6 levels (FIG. 4A), a reduction of plasma IFN alpha (FIG. 4B), a reduction of plasma IFN-gamma (FIG. 4C), increased survival (FIG. 4D) and increased transgene expression (FIG. 4E), as compared to WT mice. ULOQ indicates upper limit of quantitation. LLOQ refers to lower limit of quantitation.

### DETAILED DESCRIPTION OF THE INVENTION

The present invention features methods and compositions for intracellular DNA delivery to a subject employing a nanoparticle comprising the DNA, and a cytosolic DNA-sensing inhibitor selected from the group consisting of a cGAS-STING pathway inhibitor and an inflammasome pathway inhibitor. Intracellular DNA delivery has different uses including delivery of a DNA vector into a subject for transgene expression. Potential benefits of inhibiting the cGAS-STING pathway and/or an inflammasome pathway include reduced sensitivity to an inflammatory response due to a DNA payload.

A “nanoparticle” refers to a small non-viral particle that can encapsulate or associate with DNA and facilitates DNA delivery to a cell. The nanoparticle may also be used to deliver, for example, different DNA vectors, different transgenes, cytosolic DNA-sensing inhibitors, and immune cell modulators. The nanoparticle ranges in size from about 10 nm to about 1000 nm. In different embodiments, the nanoparticle is about 50 nm to about 500 nm, or about 50 nm to about 200 nm.

Reference to “subject” indicates a mammal, including humans; non-human primates such as apes, gibbons, gorillas, chimpanzees, orangutans, macaques; domestic animals, such as dogs and cats; farm animals such as poultry and ducks, horses, cows, goats, sheep and pigs; and experimental animals such as mice, rats, rabbits, guinea pigs. A preferred subject is a human subject being treated. However, a subject can also include animal disease models, for example, mouse and other animal models of protein/enzyme deficiencies such as Pompe disease (loss of GAA), and glycogen storage diseases (GSDs).

References to “DNA vector” indicates a DNA sequence containing a transgene operative linked to one more regulatory element providing for RNA expression from the transgene. The produced RNA can itself be functional or can encode for a protein. One type of regulatory element is a promoter, which binds RNA polymerase and the necessary transcription factors to initiate transcription. When encoding for protein, the produced RNA sequence will also encode a termination sequence at the end of the coding sequence. Additional regulatory elements include those impacting RNA expression, RNA stability, and protein production. DNA vectors may be single-stranded, double-stranded, or contain a combination of single and double stranded regions. The DNA vector may also include more than transgene and multiple regulatory elements of the same or different types.

The term “operatively linked” refers to the association of two or more nucleic acid segments on a single nucleic acid where the function of one is affected by the other.

Reference to “transgene” indicates a DNA region capable of being expressed to RNA, without regard to origin of the polynucleotide sequence. The transgene is generally part of a longer length nucleic acid, where the nucleic acid contains at least one region with which the transgene is not normally associated with in nature.

A cytosolic DNA-sensing inhibitor selected “from the group consisting of” provides that at least one member of the group is present and does not exclude, for example, both members of the group being present or the presence of one or more additional inhibitors.

A first aspect of the invention describes a method of intracellular delivery of a DNA to a subject comprising the steps of administering:
a) a cytosolic DNA-sensing inhibitor selected from the group consisting of a cGAS-STING pathway inhibitor and an inflammasome pathway inhibitor; and
b) a first nanoparticle comprising the DNA; wherein step (b) is performed prior to, concomitantly with, or after step (a).

A second aspect of the invention describes a method of intracellular delivery of a DNA to a subject comprising the steps of administering:
a) a cytosolic DNA-sensing inhibitor selected from the group consisting of a cGAS STING pathway inhibitor and an inflammasome pathway inhibitor; and
b) a first nanoparticle comprising the DNA; provided that if the first nanoparticle is a lipid nanoparticle at least one of (i) the cytosolic DNA-sensing inhibitor is at least the inflammasome pathway inhibitor; (ii) the lipid nanoparticle does not comprise an endosomolytic agent; (iii) the DNA is circular; (iv) the DNA is not closed-ended DNA; or (v) the cytosolic DNA-sensing inhibitor is provided in a second nanoparticle, wherein the second nanoparticle may have the same or different composition as the first nanoparticle; wherein step (b) is performed prior to, concomitantly with, or after step (a).

Stanton and Manganiello International Patent Publication No. WO2020/181168, mentions different endosomolytic agents and the use LNPs with an endosomolytic agent and closed-ended DNA.

In a first embodiment of the first and second aspects, the DNA is a DNA vector comprising a transgene operatively linked to a regulatory element. In further embodiments the transgene is operatively linked to a promoter; is operatively linked to a promoter/enhancer; is operatively linked to promoter/enhancer, a polyadenylation and termination signal, and/or a regulatable element; and the DNA vector comprises 5′ to 3′ the promoter/enhancer, the transgene, and the polyadenylation and termination signal.

Reference to 5′ to 3′ with respect to specified elements indicates the relative position of different elements, does not require the different elements be adjacent to each other, and allows for the presence of additional sequences. The additional sequences, which provide for additional activity, can be located in different positions such in between two identified elements, at the '3 end, or at the 5′ end.

In a second embodiment of the first and second aspects, the DNA-sensing inhibitor is a cGAS-STING pathway inhibitor; and the DNA is as provided in the first or second aspects, or the first embodiment. In further embodiments, the cGAS-STING pathway inhibitor is a cGAS inhibitor; the cGAS-STING pathway inhibitor is a STING inhibitor; the cGAS-STING pathway inhibitor is a TBK1 inhibitor; the cGAS-STING pathway inhibitor is a cGAS inhibitor provided in Section II.A. infra, the cGAS-STING pathway inhibitor is a STING inhibitor provided in Section II.A. infra and/or the cGAS-STING pathway inhibitor is TBK1 inhibitor provided in Section II.A. infra. In further embodiments the inhibitor is H-151, GSK-690693, RU-521, RO-3150, ISD 017, SI-001, CYT387 or GSK8612, or a pharmaceutically acceptable salt thereof; or is a compound from Table 1, Table 2, or Table 3, or a pharmaceutically acceptable salt thereof.

Reference to a particular embodiment includes reference to further embodiments provided therein. For example, reference in the second embodiment to the first embodiment provides a reference to all the embodiments provided in the first embodiment including the further embodiments provided therein.

In a third embodiment of the first and second aspects the cytosolic DNA-sensing inhibitor is an inflammasome pathway inhibitor, and the DNA is as provided in the first or second aspects, or the first embodiment. In further embodiments, the inflammasome pathway inhibitor is an AIM2 inhibitor; the AIM2 inhibitor is a provided in Section II.B. Infra; and the AIM2 inhibitor is A151.

In a fourth embodiment of the first and second aspects, DNA is a DNA vector comprising a transgene encoding a viral antigen, a bacterial antigen, a therapeutic protein, a short hair pin RNA (shRNA), a small interfering RNA (siRNA), a microRNA (miRNA), a RNAᵢ, a ribozyme, an antisense RNA, a clustered regularly interspaced short palindromic repeats (CRISPR)/Cas9 construct, a zinc finger nuclease (ZFN), or a transcription activator-like effector nuclease (TALEN); where the DNA may be as provided in the first embodiment; the cytosolic DNA-sensing inhibitor is as provided in the first or second aspects, the second embodiment or the third embodiment. In further embodiments the therapeutic protein is as provided in Section III.D. infra.

In a fifth embodiment, the DNA provided in any of the first or second aspects or related embodiments is circular DNA. Reference to “related embodiments” indicates each of the provided embodiments (including further embodiments) referred to with respect to a particular aspect.

In a sixth embodiment of the first and second aspects, the cytosolic DNA-sensing inhibitor is provided in a second nanoparticle; the DNA is as provided in the first or second aspects or the first, fourth or fifth embodiments; the cytosolic DNA-sensing inhibitor is as provided in the first or second aspects, the second embodiment or the third embodiments. In further embodiments, the second nanoparticle has substantially the same composition as the first nanoparticle; and the first and second nanoparticles are lipid nanoparticles or lipid polymer nanoparticles.

In a seventh embodiment of the first and second aspects, the cytosolic DNA-sensing inhibitor is provided along with the DNA in the first nanoparticle; the DNA is as provided in the first or second aspects or the first, fourth or fifth embodiment; and the cytosolic DNA-sensing inhibitor is as provided in the first or second aspects, the second embodiment or the third embodiment. In further embodiments the first nanoparticle is a lipid nanoparticle; the first nanoparticle is a lipid polymer nanoparticle; the first nanoparticle is an exosome; the first nanoparticle is configured to release the inhibitor prior to the release of the DNA; the first nanoparticle is lipid nanoparticle configured to release the inhibitor prior to the release of the DNA; the first nanoparticle is a lipid polymer nanoparticle configured to release the inhibitor prior to the release of the DNA; and the first nanoparticle is a exosome configured to release the inhibitor prior to the release of the DNA.

In an eighth embodiment of the first and second aspects, and related embodiment, the cytosolic DNA-sensing inhibitor is administered at about the same time, prior to or after administration of the DNA. In different embodiments, the cytosolic DNA-sensing inhibitor is administered at least 30 minutes, at least 60 minutes, at least 90 minutes, or at least 120 minutes prior to the DNA; the cytosolic DNA-sensing inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, up to about 4 hours, up to about 5 hours, up to about 6 hours, up to about 7 hours, up to about 8 hours, up to about 9 hours, up to about 10 hours, up to about 12 hours, up to about 1 day, up to about 2 days, up to about 3 days, up to about 4 days, or up to about a week prior to DNA administration. Preferably, the cytosolic DNA-sensing inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, or up to about 4 hours prior to DNA vector administration. In different embodiments, the cytosolic DNA-sensing inhibitor is administered at least 15 minutes, at least 30 minutes, at least 45 minutes, at least 60 minutes, at least 90 minutes, at least 120 minutes, or at least 1 day after the DNA.

In a ninth embodiment of the first and second aspects, two different cytosolic DNA-sensing inhibitors are used; where the other components and administration are as provided in the first aspect, the second aspect, or related embodiments. The different inhibitors can be provided without a nanoparticle, can be provided in different nanoparticles, or can be provided in the same nanoparticle. In further embodiments, an inflammasome pathway inhibitor and a cGAS-STING pathway inhibitor are administered; two different inflammasome pathway inhibitors are administered; and two different cGAS-STING pathway inhibitors are administered.

In a tenth embodiment of the first and second aspects or related embodiments the subject is a human patient.

In an eleventh embodiment of the first and second aspects or related embodiments the DNA and DNA vector either substantially comprise double-stranded DNA or the DNA and DNA vector substantially comprise single-stranded DNA. DNA includes double-stranded DNA, single-stranded DNA, and DNA having single and double-stranded regions. Reference to the DNA, which includes DNA making up a vector, “substantially” comprise, comprises, or comprising “double-stranded DNA” indicates more than half, at least 75%, at least 90%, at least 95%, or at least 99% of the DNA is double-stranded DNA or 100% of the DNA is double-stranded. Similarly, reference to the DNA, which includes DNA making up a vector, “substantially” comprise, comprises, or comprising “single-stranded DNA” indicates more than half, at least 75%, at least 90%, at least 95%, or at least 99% of the DNA is single-stranded DNA or 100% of the DNA is single-stranded.

In twelfth embodiment of the first and second aspects or related embodiments, the nanoparticle is an LNP. In further embodiments the LPN in mol % comprises, consists essentially, or consists, of the following components: (1) one or more cationic lipids from about 20% to 65%, one or more phospholipid lipids from about 1% to about 50%, one or more PEG-conjugated lipid from about 0.1% to 10%, and cholesterol from about 0% to about 70%; and (2) one or more cationic lipids from about 20% to 50%, one or more phospholipid lipids from about 5% to about 20%, one or more PEG-conjugated lipids from about 0.1% to 5%, and cholesterol from about 20% to about 60%. In further embodiments the phospholipid lipid is a neutral lipid; and the phospholipid lipid is DOPE or DSPC.

In a thirteenth embodiment of the first and second aspects or related embodiments the LNP, in mole %, comprises, consists essentially, or consists of the following components: (1) cKK-E12 (further described in I.A. infra.), about 35%; C14-PEG2000, about 2.5%; cholesterol, about 46.5%; and DOPE, about 16%; (2) bCKK-E12 (further described in I.A. infra.), about 35%; C14-PEG2000, about 2.5%; cholesterol, about 46.5%; and DOPE, about 16%; (3) Lipid 9 (further described in Sabnis et al. and I.A. infra.), about 50%; C14-PEG2000, about 1.5%; cholesterol, about 38.5%; and DSPC about 10%; or (4) Lipid 5 (further described in Sabnis et al. and I.A. infra.), about 50%; C14-PEG2000 about 1.5%; cholesterol about 38.5%; and DSPC about 10%; and (5) ionizable lipid, about 50%; DSPC, about 10%; cholesterol, about 37.5%; and stabilizer (PEG-Lipid), about 2.5%; or (6) is GenVoy-ILM™ LNP (Precision NanoSystems).

A third aspect of the present invention features a nanoparticle composition comprising:
a) a DNA; and
b) a cytosolic DNA-sensing inhibitor selected from the group consisting of a GAS-STING pathway inhibitor and an inflammasome pathway inhibitor.

A fourth aspect of the present invention features a nanoparticle comprising:
a) A DNA; and
b) a cytosolic DNA-sensing inhibitor selected from the group consisting of a cGAS-STING pathway inhibitor and an inflammasome pathway inhibitor, provided that if the nanoparticle is a lipid nanoparticle then at least one of (i) the cytosolic DNA-sensing inhibitor is at least the inflammasome pathway inhibitor; (ii) the lipid nanoparticle does not comprise an endosomolytic agent; (iii) the DNA is circular; (iv) the DNA is not closed-ended DNA; or (v) the cytosolic DNA-sensing inhibitor is provided in a second nanoparticle, wherein the second nanoparticle may have the same or different composition as the first nanoparticle.

In a first embodiment of the third and fourth aspects, the DNA is a DNA vector comprising a transgene, wherein the transgene comprises a promoter operatively linked to a regulatory element. In further embodiments, the transgene is operatively linked to a promoter; is operatively linked to a promoter enhancer; is operatively linked to promoter/enhancer, a polyadenylation and termination signal and/or regulatable element; and the DNA vector comprises 5′ to 3′ the promoter/enhancer, the transgene, and the polyadenylation and termination signal.

In a first embodiment of the third and fourth aspects, the DNA is a DNA vector comprising a transgene operatively linked to a regulatory element. In further embodiments the transgene is operatively linked to a promoter; is operatively linked to a promoter/enhancer; is operatively linked to promoter/enhancer, a polyadenylation and termination signal, and/or a regulatable element; and the DNA vector comprises 5′ to 3′ the promoter/enhancer, the transgene, and the polyadenylation and termination signal.

In a second embodiment of the third and fourth aspects, the cytosolic DNA-sensing inhibitor is a cGAS-STING pathway inhibitor; and the DNA is as provided in the third or fourth aspects or the first embodiment. In further embodiments, the cGAS-STING pathway inhibitor is a cGAS inhibitor; the cGAS-STING pathway inhibitor is a STING inhibitor; the cGAS-STING pathway inhibitor is a TBK1 inhibitor; the cGAS-STING pathway inhibitor is a cGAS inhibitor provided in Section II.A. infra; the cGAS-STING pathway inhibitor is a STING inhibitor provided in Section II.A. infra; and/or the cGAS-STING pathway inhibitor is TBK1 inhibitor as provided in Section II.A. infra. In further embodiments, the inhibitor is H-151, GSK-690693, RU-521, RO-3150, ISD 017, SI-001, CYT387 or GSK8612, or a pharmaceutically acceptable salt thereof; or a compound of Table 1, Table 2, or Table 3, or a pharmaceutically acceptable salt thereof.

In a third embodiment of the third and fourth aspects, the inhibitor is an inflammasome pathway inhibitor; and the DNA is as provided in the third or fourth aspects or the first embodiments. In further embodiments, the inflammasome pathway inhibitor is an AIM2 inhibitor; the AIM2 inhibitor is a provided in Section II.B. Infra; and the AIM2 inhibitor is A151.

In a fourth embodiment of the third and fourth aspects, the DNA is a DNA vector comprising a transgene encoding a viral antigen, a bacterial antigen, a therapeutic protein, a short hair pin RNA (shRNA), a small interfering RNA (siRNA), a microRNA (miRNA), a RNAᵢ, a ribozyme, an antisense RNA, a clustered regularly interspaced short palindromic repeats (CRISPR)/Cas9 construct, a zinc finger nuclease (ZFN), or a transcription activator-like effector nuclease (TALEN); where the DNA vector is as provided in the first embodiment; the cytosolic DNA-sensing inhibitor is as provided in the third or fourth aspects, the second embodiment or the third embodiment. In further embodiments the therapeutic protein is as provided in Section III.D. infra.

In a fifth embodiment the DNA is as provided in any of the third or fourth aspects, or related embodiments, is circular DNA.

In a sixth embodiment of the third and fourth aspects, the nanoparticle is a lipid nanoparticle, a lipid polymer nanoparticle, or an exosome; the DNA is as provided in the third or fourth aspects or the first, fourth or fifth embodiments; the cytosolic DNA-sensing inhibitor is as provided in the third or fourth aspects, or the second or third embodiments. In further embodiments the nanoparticle is a lipid nanoparticle; the nanoparticle is a lipid polymer nanoparticle; the nanoparticle is an exosome; the nanoparticle is lipid nanoparticle configured to release the inhibitor prior to the release of the DNA; the nanoparticle is a lipid polymer nanoparticle configured to release the inhibitor prior to the release of the DNA; and the nanoparticle is an exosome configured to release the inhibitor prior to the release of the DNA.

In further embodiments, the nanoparticle is an LNP. In further embodiments the LPN in mol % comprises, consists essentially, or consists, of the following components: (1) one or more cationic lipids from about 20% to 65%, one or more phospholipid lipids from about 1% to about 50%, one or more PEG-conjugated lipid from about 0.1% to 10%, and cholesterol from about 0% to about 70%; and (2) one or more cationic lipids from about 20% to 50%, one or more phospholipid lipids from about 5% to about 20%, one or more PEG-conjugated lipids from about 0.1% to 5%, and cholesterol from about 20% to about 60%. In further embodiments the phospholipid lipid is a neutral lipid; and the phospholipid lipid is DOPE or DSPC.

In further embodiments the LNP, in mole %, comprises, consists essentially, or consists of the following components: (1) cKK-E12 (further described in I.A. infra.), about 35%; C14-PEG2000, about 2.5%; cholesterol, about 46.5%; and DOPE, about 16%; (2) bCKK-E12 (further described in I.A. infra.), about 35%; C14-PEG2000, about 2.5%; cholesterol, about 46.5%; and DOPE, about 16%; (3) Lipid 9 (further described in Sabnis et al. and I.A. infra.), about 50%; C14-PEG2000, about 1.5%; cholesterol, about 38.5%; and DSPC about 10%; or (4) Lipid 5 (further described in Sabnis et al. and I.A. infra.), about 50%; C14-PEG2000 about 1.5%; cholesterol about 38.5%; and DSPC about 10%; and (5) ionizable lipid, about 50%; DSPC, about 10%; cholesterol, about 37.5%; and stabilizer (PEG-Lipid), about 2.5%; or (6) is Gen Voy-ILM™ LNP (Precision NanoSystems).

In a seventh embodiment of the third and fourth aspects, two different cytosolic DNA-sensing inhibitors are provided in the nanoparticle; where the other components are as provided in the third or fourth aspects or related embodiments. In further embodiments, an inflammasome pathway inhibitor and a cGAS-STING pathway inhibitor are provided; two different inflammasome pathway inhibitors are provided; and two different cGAS-STING pathway inhibitors are provided.

In an eighth embodiment of the third and fourth aspects or related embodiments the DNA and DNA vector each substantially comprise double-stranded DNA; and the DNA and DNA vector each substantially comprise single-stranded DNA.

A fifth aspect is directed to a pharmaceutical composition comprising the nanoparticle composition of the third or fourth aspects or related embodiments and a pharmaceutically acceptable carrier.

A sixth aspect is directed to a pharmaceutical composition for use in gene therapy comprising a DNA vector comprising a transgene wherein the composition is for use prior to, concomitantly with, or after administration of a cytosolic DNA-sensing inhibitor, wherein the DNA vector is provided in a nanoparticle. Additional embodiments are provided in the methods described in the first and second aspects and related embodiments; and the compositions described in the third and fourth aspects and related embodiments.

A seventh aspect of the invention is directed to a method of making medicament for use in the first and second aspects and related embodiments. The medicament is made by combining the nanoparticle with a pharmaceutical acceptable carrier, where the nanoparticle is as described the first, second, third, or fourth aspects or related embodiments.

The singular forms “a,” “an,” and “the” include plural reference unless the context clearly dictates otherwise.

As used herein, the conjunctive term “and/or” between multiple recited elements is understood to encompass both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,”, “such as” followed by different members or examples, are open-ended descriptions where the listed members or examples are illustrative and other member or examples can be provided or used.

The terms “polypeptides,” “proteins” and “peptides” can be used interchangeably to refer to an amino acid sequence without regard to function. Polypeptides and peptides contain at least two amino acids, while proteins contain at least about 10 amino acid acids. The provided amino acids include naturally occurring amino acids and amino acids provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein reference to an open-ended term such as “comprising” can be replaced by the terms “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100%, as well as 95.1%, 95.2%, 95.3%, 95.4%, 95.5%, etc., 96.1%, 96.2%, 96.3%, 96.4%, 96.5% and so forth; reference to a numerical range, such as “1-4” includes 2, 3, as well as 1.1, 1.2, 1.3, 1.4 and so forth; reference to “1 to 4 weeks” includes 7, 8, 9, 10, 11, 12, 13, 14, 15, 16, 17, 18, 19, 20, 21, 22, 23, 24, 25, 26, 27, or 28 days; reference to a numerical range, such as “0.01 to 10” includes 0.011, 0.012, 0.013 and so forth, as well as 9.5, 9.6, 9.7, 9.8, 9.9 and 10 and so forth. For example, a dosage of “0.01 mg/kg to 10 mg/kg” body weight of a subject includes 0.011 mg/kg, 0.012 mg/kg, 0.013 mg/kg, 0.014 mg/kg, 0.015 mg/kg and so forth as well as 9.5 mg/kg, 9.6 mg/kg, 9.7 mg/kg, 9.8 mg/kg, 9.9 mg/kg and so forth.

Reference to an integer with more (greater) or less than includes numbers greater or less than the reference number, respectively. Thus, for example, reference to more than 2 includes 3, 4, 5, 6, 7, 8, 9, 10, 11, 12, 13, 14, 15; and administration “two or more” times includes 3, 4, 5, 6, 7, 8, 9, 10, 11, 12, 13, 14, 15, or more times.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

While certain combination of features is highlighted herein, all of the features disclosed herein can be combined in any combination. Each feature disclosed in the specification can be replaced by an alternative feature serving a same, equivalent, or similar purpose.

### I. Nanoparticles

A variety of different nanoparticles can be employed include including lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. (See, e.g., Riley and Vermerris Nanomaterials 2017, 7, 94; Thomas et al., Molecules 2019, 24, 3744; Bochicchio et al., Pharmaceutics 2021, 13, 198; Munagala et al., Cancer Letters 2021, 505, 58; Fu et al., 2020 NanoImpact 20, 100261; and Neshat et al. 2020 Current Opin. Biotechnol. 66:1-10.)

If desired, a nanoparticle can be targeted to a cell type using, for example, targeting ligands recognizing a target cell receptor. Examples of targeting ligands include carbohydrates (e.g., galactose, mannose, glucose, and galactomannan), endogenous ligands (e.g., folic acid and transferrin), antibodies (e.g., anti-HER2 antibody and hD1) and protein/peptides (e.g., RGD, epidermal growth factor, and low density lipoprotein) and peptides. (For example, Teo et al., Advanced Drug Delivery Reviews 2016, 98, 41.)

The present application features the use of nanoparticles to deliver DNA. In different embodiments, nanoparticles can deliver additional compounds such as a cytosolic DNA-sensing inhibitors, immunosuppressants, phagocyte depleting compounds, and additional therapeutic compounds; one or more additional compounds is provided in different nanoparticles; and one or more additional compounds is provided in the same nanoparticle as the DNA vector, for example a DNA vector and a cytosolic DNA sensing inhibitor or a DNA vector, a cytosolic DNA-sensing inhibitor and an immune cell modulator. Reference to “compounds” includes small molecules and large molecules (e.g., therapeutic proteins and antibodies), and nucleic acid.

The production of different nanoparticles and incorporation of nucleic acid and other compounds is well known in the art, and exemplified by different publications throughout the discussion in Section I. In general, exposure kinetics of nanoparticle cargoes (e.g., the DNA and/or inhibitor) can be affected by providing DNA and different compounds with different environments or association with different structures.

Examples of publications illustrating incorporation of nucleic acid in a particular nanoparticle such as an LPNP and a LNP include Teo et al., Advanced Drug Delivery Reviews 2016, 98, 41; Bochicchio et al., Pharmaceutics 2021, 13, 198; Mahzabin and Das, IJPSR 2021, 12 (1), 65; and Teixeira et al., Progress in Lipid Research 2017, 1 (each of which are hereby incorporated by reference herein in their entirety.). Such references also point out an advantage of LPNP in providing different structures interacting with nucleic acid and small molecules that can impact desired release kinetics. Factors that may impact small molecule incorporation into a nanoparticle include hydrophobicity and the presence of an ionizable moiety. (See, e.g., Nii and Ishii, International Journal of Pharmaceutics 2005, 298, 198; and Chen et al., Journal of Controlled Release 2018, 286, 46.)

In an embodiment, a compound (e.g., cytosolic DNA-sensing inhibitor and/or immune cell modulator) is linked to a fatty acid to increase hydrophobicity. Examples of fatty acids that can linked to small molecules include those described by Chen et al., Journal of Controlled Release 2018, 286, 46-54.

### I.A. Lipid-Based Delivery Systems

Lipid-based delivery systems include the use of a lipid as a component. Examples of lipid-based delivery systems include liposomes, LNPs, micelles, and extracellular vesicles.

A “lipid nanoparticle” or “LNP” refers to a lipid-based vesicle useful for delivery of nucleic acid molecules and having dimensions on the nanoscale. In different embodiments the nanoparticle is from about 10 nm to about 1000 nm, about 50 nm to about 500 nm, or about 50 nm to about 200 nm.

DNA is negatively charged. Thus, it can be beneficial for the LNP to comprise a cationic lipid such as, for example, an amino lipid. Exemplary amino lipids are described in U.S. Pat. Nos. 9,352,042, 9,220,683, 9,186,325, 9,139,554, 9,126,966 9,018,187, 8,999,351, 8,722,082, 8,642,076, 8,569,256, 8,466, 122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids described in U.S. Pat. No. 9,512,073, hereby incorporated herein in its entirety.

The terms “cationic lipid” and “amino lipid” are used interchangeably herein to include lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipid can also be titratable cationic lipids. In certain embodiments, the cationic lipids comprise a protonatable tertiary amine (e.g., pH-titratable) group; C18 alkyl chains, wherein each alkyl chain independently can have one or more double bonds, one or more triple bonds; and ether, ester, or ketal linkages between the head group and alkyl chains.

Cationic lipids include 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino) butanoate (DLin-M-C3-DMA, also known as MC3), salts thereof, and mixtures thereof. Other cationic lipids also include 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids include 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino) acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino) propane (DLin-MPZ), 3-(N,N-dilinoleylamino)-1,2-propanediol (DLinAP), 3-(N,N-dioleylamino)-1,2-propanedio(DOAP), 1,2-dilinoleyloxo-3-(2-N,N-dimethylamino) ethoxypropane (DLin-EG-DMA), N,N-dioleyl-N,N-dimethylammonium chloride (DODAC), N-(1-(2,3-dioleyloxy) propyl)-N,N,N-trimethylammonium chloride (DOTMA), N,N-distearyl-N,N-dimethylammonium bromide (DDAB), N-(1-(2,3-dioleoyloxy) propyl)-N,N,N-trimethylammonium chloride (DOTAP), 3-(N—(N′,N′-dimethylaminoethane)-carbamoyl) cholesterol (DC-Chol), N-(1,2-dimyristyloxyprop-3-yl)-N,N-dimethyl-N-hydroxyethyl ammonium bromide (DMRIE), 2,3-dioleyloxy-N-[2 (spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy) propane (CLinDMA), 2-[5′-(cholest-5-en-3-beta-oxy)-3′-oxapentoxy)-3-dimethyl-1-(cis,cis-9′, 1-2′-octadecadienoxy) propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Additional ionizable lipids that can be used include C12-200, 3060i10, MC3, cKK-E12, bCKK-E12, Lipid 5, Lipid 9, ATX-002, ATX-003, and Merck-32. U.S. Patent Application Publication No. 2017/0367988, describes Merck-32.

In further embodiments, cationic lipid can be present in an amount from about 10% by molar ratio of the LNP to about 85% by molar ratio of the LNP, or from about 50% by molar ratio of the LNP to about 75% by molar ratio of the LNP.

LNP can comprise a neutral lipid. Neutral lipids can comprise a lipid species existing either in an uncharged or neutral zwitterionic form at physiological pH. Such lipids include diacylphosphatidylcholine, diacylphosphatidylethanolamine, ceramide, sphingomyelin, dihydrosphingomyelin, cephalin, and cerebrosides. The selection of neutral lipids is generally guided by considerations including particle size and stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized. In certain embodiments, lipids containing saturated fatty acids with carbon chain lengths in the range of C14 to C22 can be used. In certain embodiments lipids with mono or di-unsaturated fatty acids with carbon chain lengths in the range of C14 to C22 are used. Additionally, lipids having mixtures of saturated and unsaturated fatty acid chains can be used. Exemplary neutral lipids include 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or a phosphatidylcholine. The neutral lipids can also be composed of sphingomyelin, dihydrosphingomyelin, or phospholipids with other head groups, such as serine and inositol.

In further embodiments, providing for neutral lipids, the neutral lipid can be present in an amount from about 0.1% by weight of the LNP to about 99% by weight of the LNP, or from about 5% by weight of the LNP to about 15% by weight of the LNP, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNP can contain additional components such as sterols and polyethylene glycol. Sterols can confer fluidity to the LNP. As used herein “sterol” refers to a naturally occurring sterol of plant (phytosterols) or animal (zoosterols) origin as well as non-naturally occurring synthetic sterols, all of which are characterized by the presence of a hydroxyl group at the 3-position of the steroid A-ring. Suitable sterols include those conventionally used in the field of liposome, lipid vesicle or lipid particle preparation, most commonly cholesterol. Phytosterols include campesterol, sitosterol, and stigmasterol. Sterols also include sterol-modified lipids, such as those described in U.S. Patent Application Publication 2011/0177156. In different embodiments providing for a sterol, the sterol is present in an amount from about 1% by weight of the LNP to about 80% by weight of the LNP or from about 10% by weight of the LNP to about 25% by weight of the LNP.

Polyethylene glycol (PEG) is a linear, water-soluble polymer of ethylene PEG repeating units with two terminal hydroxyl groups. PEGs are classified by their molecular weights, for example, PEG 2000 has an average molecular weight of about 2,000 daltons, and PEG 5000 has an average molecular weight of about 5,000 daltons. PEGs commercially available from Sigma Chemical Co. and other companies include monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments concerning PEG, PEG has an average molecular weight of about 550 to about 10,000 daltons and is optionally substituted by alkyl, alkoxy, acyl or aryl. In further embodiments, the PEG is substituted with methyl at the terminal hydroxyl position. In further embodiments, the PEG has an average molecular weight from about 750 to about 5,000 daltons, or from about 1,000 to about 5,000 daltons, or from about 1,500 to about 3,000 daltons, or from about 2,000 daltons, or from about 750 daltons.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.1% by weight of the LNP to about 50% by weight of the LNP, or from about 5% by weight of the LNP to about 15% by weight of the LNP.

In further embodiments concerning LNP size, prior to encapsulating nucleic acid, LNPs have a size range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or Billingsley et al., International Patent Publication No. WO 2021/077066 (both of which are hereby incorporated by reference herein in their entirety). Billingsley et al., and WO2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

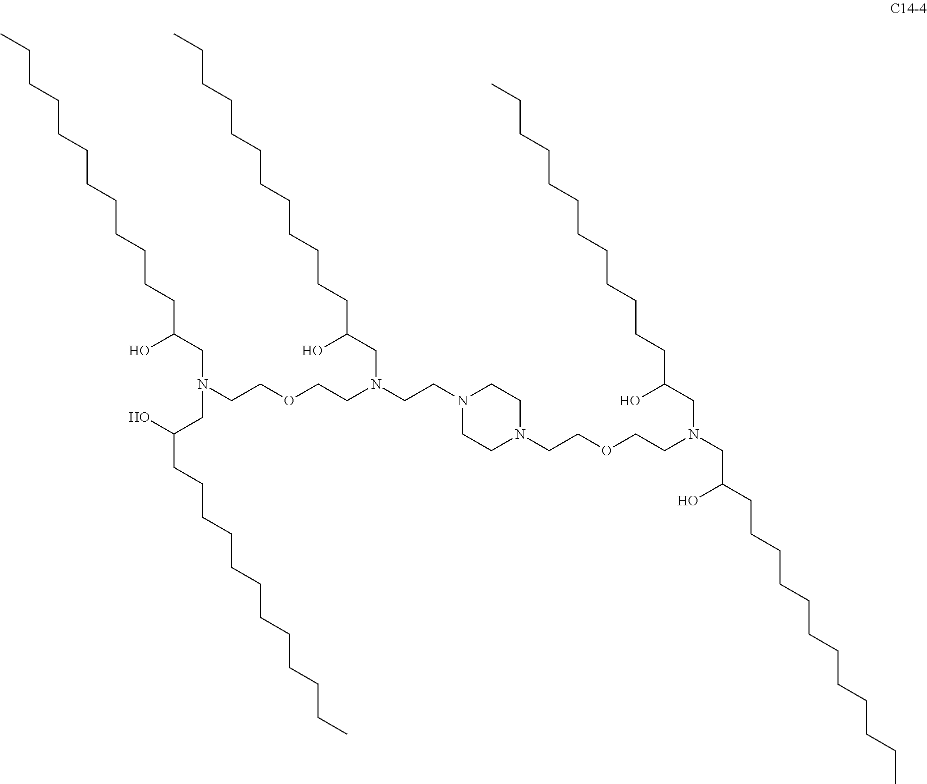 [SMILES: CCCCCCCCCCCCC(O)CN(CCOCCN(CC(O)CCCCCCCCCCCC)CC(O)CCCCCCCCCCCC)CCN1CCN(CCOCCN(CC(O)CCCCCCCCCCCC)CC(O)CCCCCCCCCCCC)CC1]

In certain embodiments the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipids. In certain embodiments the LNP contains cKK-E12 (Dong et al., PNAS (2014) 111 (11), 3955):

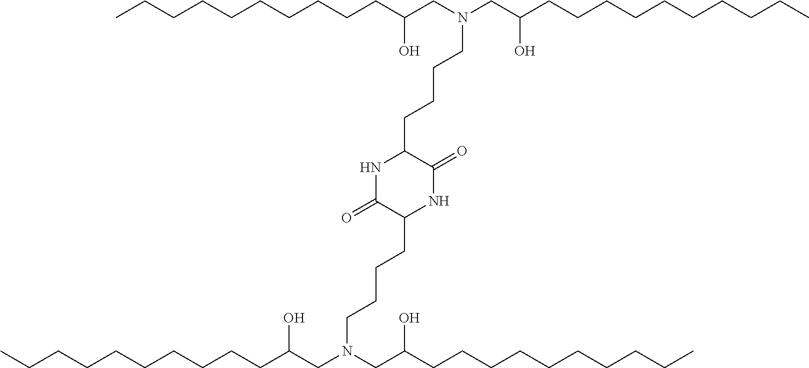 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In certain embodiments the LNP comprises a modified form of cKK-E12 referred to herein as “bCKK-E12,” having the following structure:

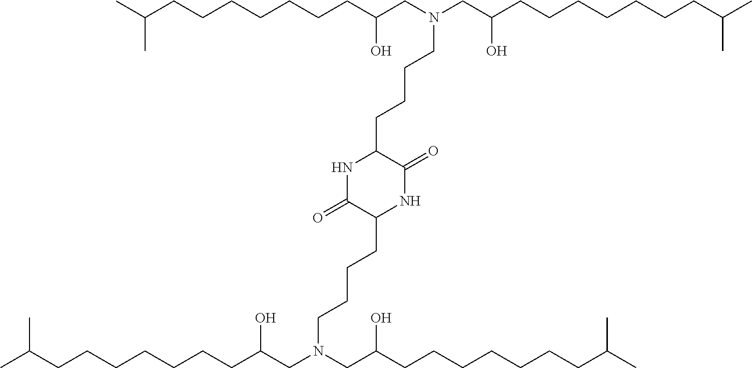 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

In certain embodiments the LNP comprises Lipid 1, 2, 3, 4, 5, 6, 7, 8, 9, or 10 as described by Sabnis et al., Molecular Therapy 2018, 26:6, 1509-1519 (hereby incorporated by reference herein in its entirety). In certain embodiments the LNP comprises Lipid 5, 8, 9, 10, or 11 described in Sabnis et al.

Lipid 5 of Sabnis et al. has the structure:

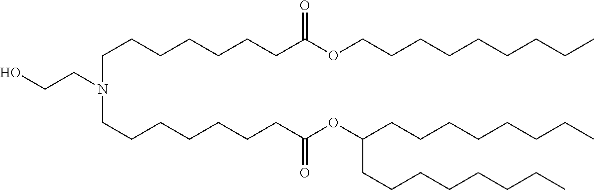 [SMILES: CCCCCCCCCOC(=O)CCCCCCCN(CCO)CCCCCCCC(=O)OC(CCCCCCCC)CCCCCCCC]

Lipid 9 of Sabnis et al. has the structure:

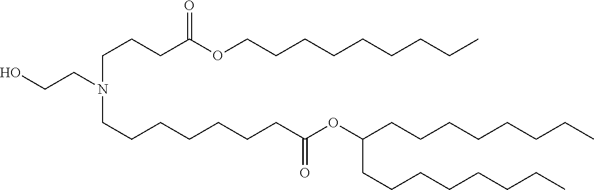 [SMILES: CCCCCCCCCOC(=O)CCCN(CCO)CCCCCCCC(=O)OC(CCCCCCCC)CCCCCCCC]

Additional lipids that may be utilized include those described by Roces et al., Pharmaceutics, 2020, 12, 1095; Jayaraman et al, Angew. Chem. Int. Ed., 2012, 51, 8529-8533; Maier et al., www.moleculartherapy.org, 2013, Vol. 21, No. 8, 1570-1578; Liu et al., Adv. Mater. 2019, 31, 1902575, e.g., BAMEA-016B; Cheng et al., Adv. Mater., 2018, 30, 1805308, e.g., 5A2-SC8; Hajj and Ball, Small, 2019 15, 1805097, e.g., 3060110; Du et al., U.S. Patent Application Publication No. 20160376224; and Tanaka et al., Adv. Funct. Mater., 2020, 30, 1910575; each of which are hereby incorporated by reference herein in their entirety.

In further embodiments, the nanoparticle is an LNP. In further embodiments the LPN in mol % comprises, consists essentially, or consists, of the following components: (1) one or more cationic lipids from about 20% to 65%, one or more phospholipid lipids from about 1% to about 50%, one or more PEG-conjugated lipid from about 0.1% to 10%, and cholesterol from about 0% to about 70%; and (2) one or more cationic lipids from about 20% to 50%, one or more phospholipid lipids from about 5% to about 20%, one or more PEG-conjugated lipids from about 0.1% to 5%, and cholesterol from about 20% to about 60%. In further embodiments the phospholipid lipid is a neutral lipid; and the phospholipid lipid is DOPE or DSPC.

In further embodiments the LNP, in mole %, comprises, consists essentially, or consists of the following components: (1) cKK-E12 (further described in I.A. infra.), about 35%; C14-PEG2000, about 2.5%; cholesterol, about 46.5%; and DOPE, about 16%; (2) bCKK-E12 (further described in I.A. infra.), about 35%; C14-PEG2000, about 2.5%; cholesterol, about 46.5%; and DOPE, about 16%; (3) Lipid 9 (further described in Sabnis et al. and I.A. infra.), about 50%; C14-PEG2000, about 1.5%; cholesterol, about 38.5%; and DSPC about 10%; or (4) Lipid 5 (further described in Sabnis et al. and I.A. infra.), about 50%; C14-PEG2000 about 1.5%; cholesterol about 38.5%; and DSPC about 10%; and (5) ionizable lipid, about 50%; DSPC, about 10%; cholesterol, about 37.5%; and stabilizer (PEG-Lipid), about 2.5%; or (6) is GenVoy-ILM™ LNP (Precision NanoSystems).

### I.B. Polymer-Based Nanoparticles

Polymer-based delivery systems can be made from a variety of different natural and synthetic materials. DNA and other compounds can be entrapped into the polymeric matrix of polymeric nanoparticles or can be adsorbed or conjugated on the surface of the nanoparticles. Examples of commonly used polymers for nucleic acid delivery include poly(lactic-co-glycolic acid) (PLGA), poly lactic acid (PLA), poly(ethylene imine) (PEI) and PEI derivatives, chitosan, dendrimers, polyanhydride, polycaprolactone, polymethacrylates, poly-L-lysine, pullulan, dextran, and hyaluronic acid, poly-β-aminoesters. (Thomas et al., Molecules 2019, 24, 3744.)

In certain embodiments, the polymeric-based nanoparticles have different sizes, ranging from about 1 nm to about 1000 nm, from about 10 nm to about 500 nm, from about 50 nm to about 200 nm, from about 100 nm to about 150 nm, and from about 150 nm or less.

### I.C. Lipid Polymer Nanoparticles

Lipid polymer nanoparticles are hybrid nanoparticles providing both a lipid component and a polymer component, and as such can be considered to be an LNP or LPNP. The LPNP configuration can provide an outer polymer and inner lipid or an outer lipid and inner polymer. The presence of two different types of material facilitates designing nanoparticles to provide for delayed release of a component. Different lipid and polymer components can be selected taking into account the material be delivered (e.g., cytosolic DNA-sensing inhibitor and DNA vector), along with guidance provided in I.A. supra., and I.B. supra., and provided in the art. (For example, Teo et al., Advanced Drug Delivery Reviews 2016, 98, 41; Bochicchio et al., Pharmaceutics, 2021 13, 198; Mahzabin and Das, IJPSR 2021, 12 (1), 65; Teixeira et al., Progress in Lipid Research, 2018, 1.)

### I.D. Protein and Peptide-Based Nanoparticles

Protein and peptide-based systems can employ a variety of different proteins and peptides. Examples of proteins include gelatin and elastin. Peptide-based systems can employ, for example, CPPs,

CPPs are short peptides (6-30 amino acid residues) potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25 (1): 1996-2006). Examples of CPPs include cationic CPPs (highly positively charged) such as the Tat peptide, penetratin, protamine, poly-L-lysine, and polyarginine; amphipathic CPPs (chimeric or fused peptides, constructed from different sources, containing both positively and negatively charged amino acid sequences), such as transportan, VT5, bactenecin-7 (Bac7), proline-rich peptide (PPR), SAP (VRLPPP)₃, TP10, pep-1, and MPG.); membranotropic CPPs (exhibit both hydrophobic and amphipathic nature simultaneously, and comprise both large aromatic residues and small residues) such as H625, SPIONs-PEG-CPP and NPs; and hydrophobic CPPs (contain only non-polar motifs or residues) such as SG3, PFVYLI, pep-7, and fibroblast growth factors.

The protein and peptide nanoparticles can be provided in different sizes for example, ranging from about 1 nm to about 1000 nm, from about 10 nm to about 500 nm, from about 50 nm to about 200 nm, from about 100 nm to about 150 nm, or from about 150 nm or less.

### I.E. Peptide Cage Nanoparticles

Peptide cage-based delivery systems can be produced from proteinaceous material able to assemble into a cage-like structure forming a constrained internal environment. Peptide cages can comprise a proteinaceous shell that self-assembles to form a protein cage (e.g., a structure with an interior cavity that is either naturally accessible to the solvent or can be made so by altering solvent concentration, pH, or equilibria ratios). The monomers of the protein cages can be naturally occurring or variant forms, including amino acid substitutions, insertions, and deletions (e.g., fragments).

Different types of protein “shells” can be assembled and loaded with different types of materials. Protein cages can be produced using viral coat protein(s) (e.g., from the Cowpea Chlorotic Mottle Virus protein coat), as well non-viral proteins (e.g., U.S. Pat. Nos. 6,180,389 and 6,984,386, U.S. Patent Application Publication No. 20040028694, and U.S. Patent Application Publication No. 20090035389, each of which is incorporated by reference herein in their entity).

Examples of protein cages derived from non-viral proteins include: eukaryotic or prokaryotic derived ferritins and apoferritins such as 12 and 24 subunit ferritins; and heat shock proteins (HSPs), such as the class of 24 subunit heat shock proteins that form an internal core space, the small HSP of Methanococcus jannaschii, the dodecameric Dsp HSP of E. coli; and the MrgA protein.

In certain embodiments, the protein cages have different core sizes, such as ranging from about 1 nm to about 1000 nm, from about 10 nm to about 500 nm, from about 50 nm to about 200 nm, from about 100 nm to about 150 nm, or from about 150 nm or less.

### I.F. Exosomes

Exosomes are small biological membrane vesicles that been utilized to deliver various cargoes including small molecules, peptides, proteins and nucleic acids. Exosomes generally range in size from about 30 nm to 100 nm and can be taken up by a cell and deliver its cargo. Cargoes can be associated with exosome surface structure or may be encapsulated within the exosome bilayer.

Various modifications can be made to exosomes facilitating cargo delivery and cell targeting. Modifications for facilitating cargo delivery include structures for associating with cargoes such as protein scaffolds and polymers. Modifications for cell targeting include targeting ligands and modifying surface charge. Publications describing production, modification, and use of exosomes for delivery of different cargoes include Munagala et al., Cancer Letters 2021, 505, 58; Fu et al., 2020 NanoImpact 20, 100261; and Dooley et al., 2021 Molecular Therapy 29 (5), 1729 (each of which is hereby incorporated by reference herein).

### II. Cytosolic DNA-Sensing Pathway Inhibitor

The cytosolic DNA-sensing pathway detects foreign DNA and produces an immune response resulting in production of proinflammatory cytokines, proinflammatory chemokines, and Type I interferons. (For example, hypertext transfer protocol://www.genome.jp/dbget-bin/www_bget?pathway+hsa04623, hereby incorporated by reference herein in its entirety.) A cytosolic DNA-sensing pathway inhibitor can be provided to reduce an immune response caused by the DNA. In different embodiments, a cGAS-STING and/or an inflammasome pathway inhibitor is employed. A particular inhibitor that inhibits more than one target can be provided as an inhibitor of each or any of the targets.

A variety of different types of compounds can inhibit production or activity of proteins involved in the cytosolic DNA-sensing pathway and can be used as inhibitors. In certain embodiments, the cytosolic DNA-sensing pathway inhibitor is a small molecule, antibody, peptide, nucleic acid, or targeted protein of a degradation agent (such as a protac or degrader).

II.A. cGAS-STING Pathway Inhibitor

cGAS-STING pathway inhibitors directly affect cGAS-STING pathway proteins such as cGAS, STING, or TBK1, or can affect an agent impacting the cGAS-STING pathway. References describing cGAS-STING pathway inhibitor design, and examples of inhibitors include Ding et al., Acta Pharmaceutica Sinica B 2020, 10 (12), 2272 (“Ding”), Fu et al., iScience 2020, 23, 101026, Konno et al., Cell Rep. 2018, 23 (24), 1112, U.S. Patent Application Publication No. 20200291001, and Haag et al., Nature, 2018, 559, 269-273 (each of which are hereby incorporated by reference herein).

Various compounds described herein including cGAS-STING pathway inhibitors can be provided as pharmaceutically acceptable salt. A “pharmaceutically acceptable salt” refers to a salt suitable for administration. Depending on the compound, pharmaceutically acceptable salts include acid addition and basic salts. Pharmaceutically acceptable acid addition salts include hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Suitable base salts include aluminum, calcium, lithium, magnesium, potassium, sodium, zinc, bismuth, and diethanolamine salts. In addition, various amino acids can be employed as pharmaceutically acceptable salts.

In certain embodiments the STING inhibitor is selected from 1-oxo-1,2,3,4-teterahydroisoquinolin-4-yl carboxylic acid (44), 9-amino-6-chloro-2-methoxyacridine (59), hydrochloroquine (60),

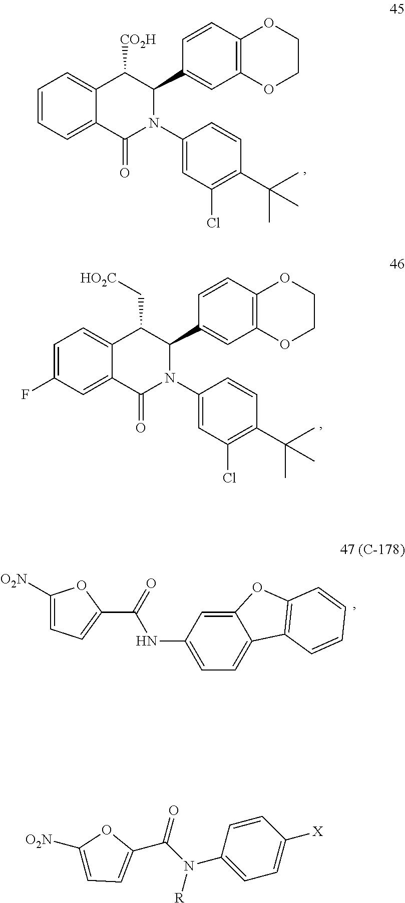

wherein R is H and X is I (48), R is H and X is n-butyl (50) or R is Hand X is n-hexyl (51),

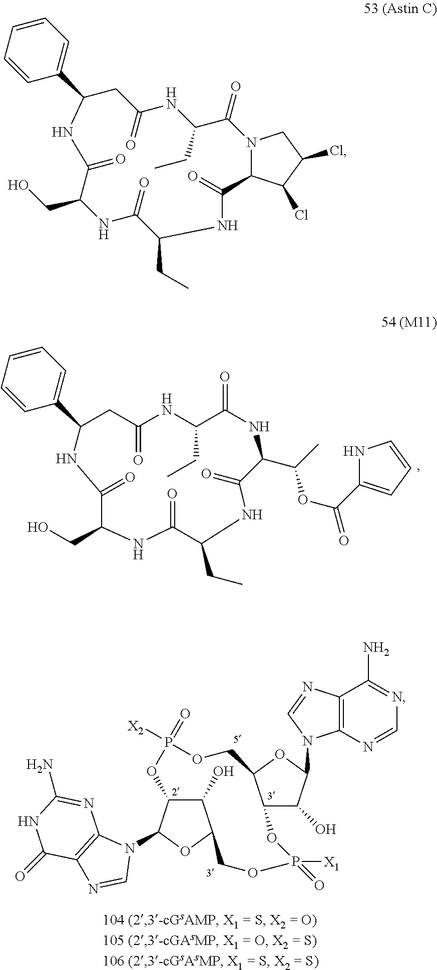

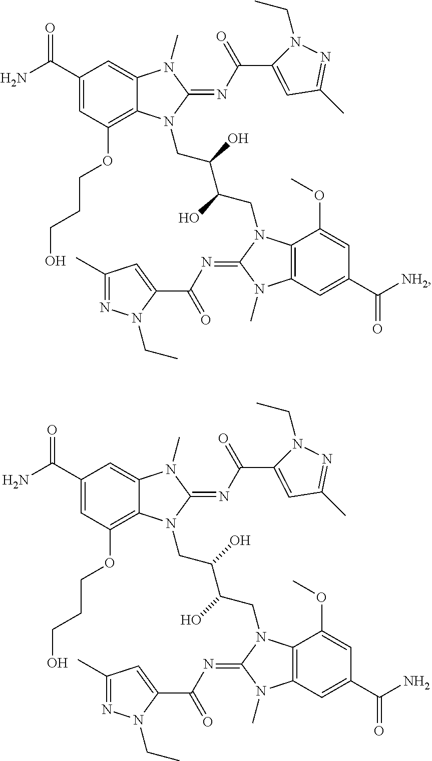 [SMILES: CCn1nc(C)cc1C(=O)/N=c1\n(C)c2cc(C(N)=O)cc(OC)c2n1C[C@@H](O)[C@H](O)Cn1/c(=N/C(=O)c2cc(C)nn2CC)n(C)c2cc(C(N)=O)cc(OCCCO)c21 ; CCn1nc(C)cc1C(=O)/N=c1\n(C)c2cc(C(N)=O)cc(OC)c2n1C[C@H](O)[C@@H](O)Cn1/c(=N/C(=O)c2cc(C)nn2CC)n(C)c2cc(C(N)=O)cc(OCCCO)c21]

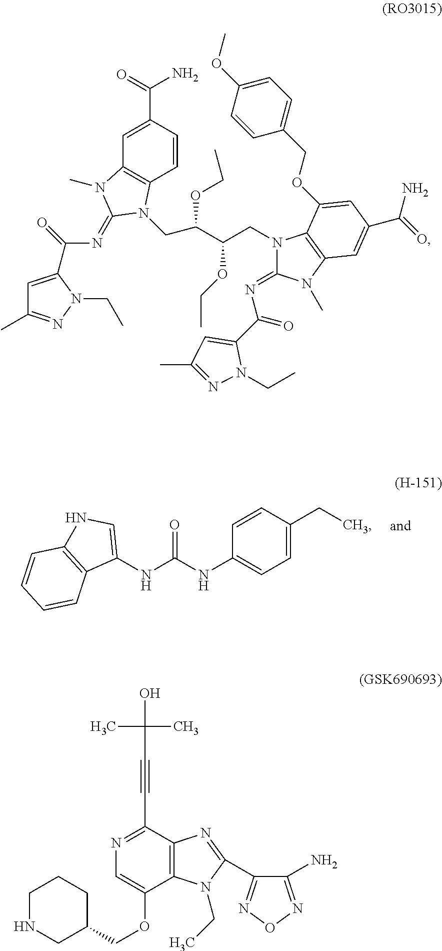 [SMILES: CCO[C@@H](Cn1/c(=N/C(=O)c2cc(C)nn2CC)n(C)c2cc(C(N)=O)ccc21)[C@H](Cn1/c(=N/C(=O)c2cc(C)nn2CC)n(C)c2cc(C(N)=O)cc(OCc3ccc(OC)cc3)c21)OCC ; CCc1ccc(NC(=O)Nc2c[nH]c3ccccc23)cc1 ; CCn1c(-c2nonc2N)nc2c(C#CC(C)(C)O)ncc(OC[C@H]3CCCNC3)c21]

or a pharmaceutically acceptable salt thereof.

GSK690693 is an AMP-activated proteins kinase (AMPK)/AKT inhibitor and provides an example of an inhibitor impacting the cGAS-STING pathway. AMPK/AKT activity impacts the cyclic cGAS-STING pathway by causing a loss in ULK1 phosphorylation, which releases ULK1 to phosphorylate STING thereby inhibiting STING activity. Examples of AMPK inhibitors, including GSK690693, are provided in Konno et al., Cell Rep. 2018, 23 (4), 1112.

Additional references providing additional STING inhibitors, STING inhibitor scaffolds and motifs, and design considerations include Decout et al., Nat. Rev. Immunol., September 2021; 21 (9): 548-569; Dubensky et al., U.S. Pat. No. 10,189,873; Katibah et al., U.S. Patent Application Publication No. 20180369268; Seidel et al., International Patent Publication No. WO2020/150439; Roush et al., U.S. Patent Application Publication No. 20200172534; Roush et al., U.S. Patent Application Publication No. 2021236466; Glick et al., International Patent Publication No. WO2022/140410; Hong et al., Proc Natl Acad. Sci USA., Jun. 15, 2021; 118 (24); and Hong et al., Journal of Molecular Cell Biology, 2022, 14 (2), njac005; each of these publications are hereby incorporated by reference herein in their entirety.

In different embodiments the inhibitor is as provided in Table 1 or a pharmaceutically acceptable salt thereof.

TABLE 1

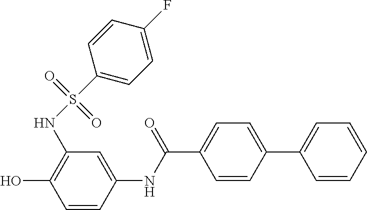 [SMILES: O=C(Nc1ccc(O)c(NS(=O)(=O)c2ccc(F)cc2)c1)c1ccc(-c2ccccc2)cc1]

SN-11

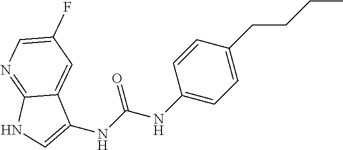 [SMILES: CCCCc1ccc(NC(=O)Nc2c[nH]c3ncc(F)cc23)cc1]

Compound 275

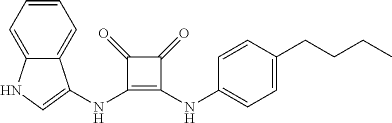 [SMILES: CCCCc1ccc(Nc2c(Nc3c[nH]c4ccccc34)c(=O)c2=O)cc1]

Compound 209

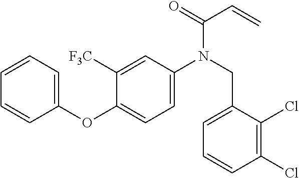 [SMILES: C=CC(=O)N(Cc1cccc(Cl)c1Cl)c1ccc(Oc2ccccc2)c(C(F)(F)F)c1]

BPK-21

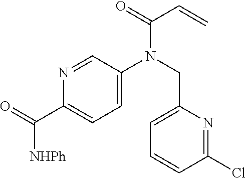

BPK-25

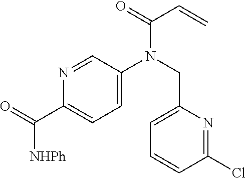

Compound 13

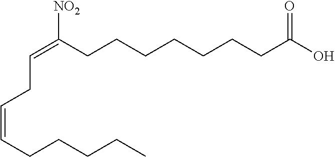 [SMILES: CCCCC/C=C\C/C=C(\CCCCCCCC(=O)O)[N+](=O)[O-]]

NO₂-cLA

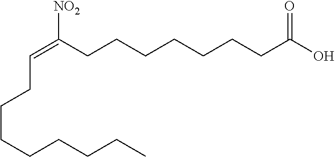 [SMILES: CCCCCCCC/C=C(\CCCCCCCC(=O)O)[N+](=O)[O-]]

NO₂-OA

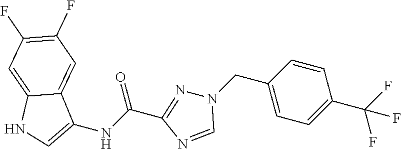 [SMILES: O=C(Nc1c[nH]c2cc(F)c(F)cc12)c1ncn(Cc2ccc(C(F)(F)F)cc2)n1]

Compound 147

In different embodiments the cGAS inhibitor is either quinacrine hydrochloride (55), ethidium bromide (56), actinomycin (57), quinacrine (58), 9-amino-6-choloro-2-methoxyacridine (59), hydroxychloroquine (60),

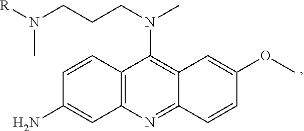

where R is

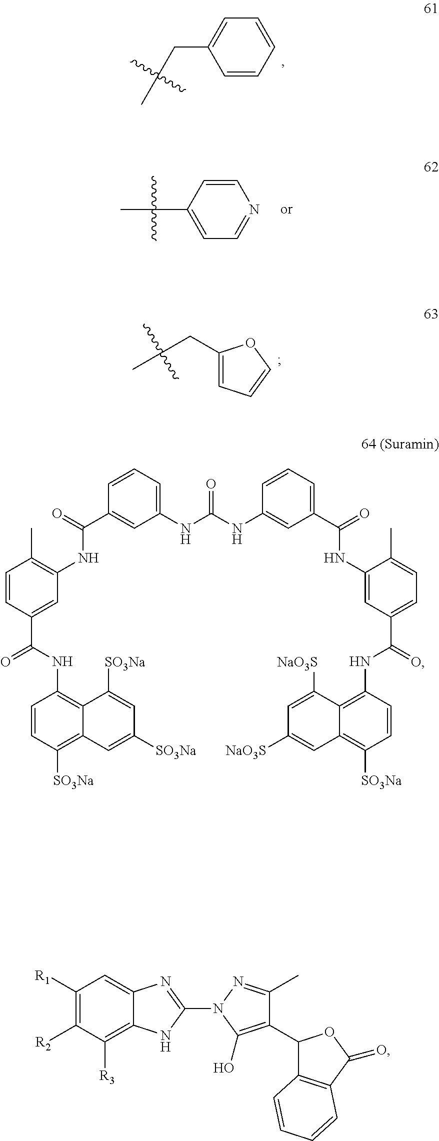

where R₁, R₂ and R₃ are each H (65); R₁ is H, and R₂ and R₅ are each C1 (66), R₁ and R₃ are each Cl and R₂ is H (67); or R₁ and R₂ are each H and R₃ is Br (68);

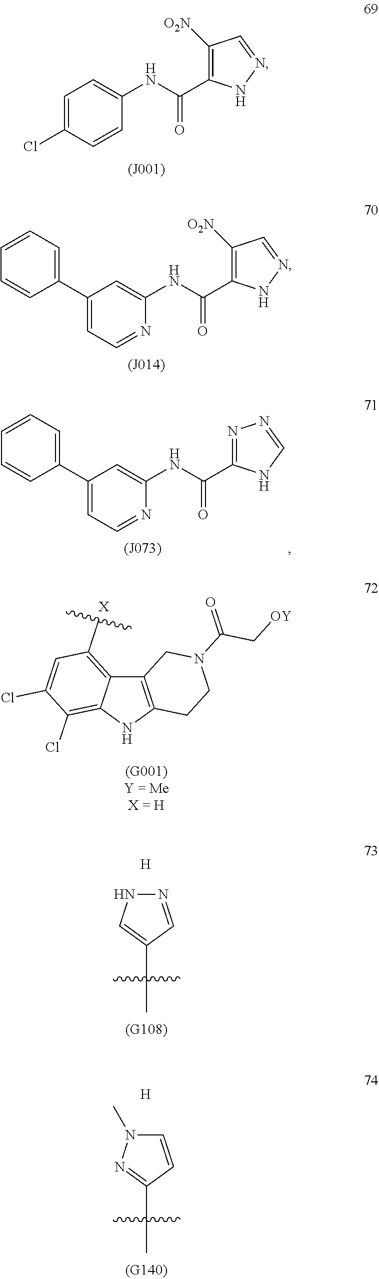

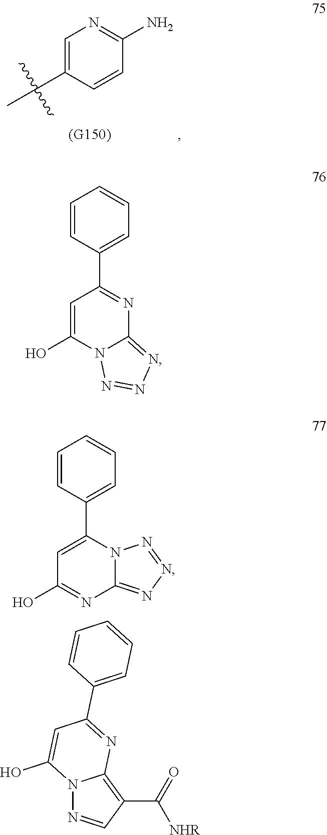

where R₁ is H (78), or R₁ is

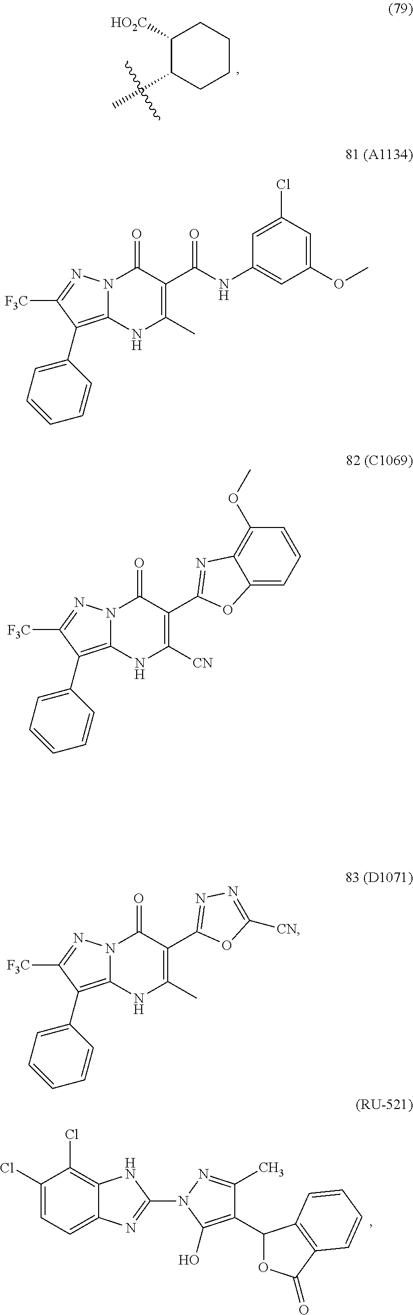

or a pharmaceutically acceptable salt thereof.

Ding describes compounds 55, 56 and 57 as indirect cGAS inhibitors that intercalate DNA and describes compounds 58 to 64 as indirect cGAS inhibitors that inhibit 2′,3′-cGAMP synthesis.

Additional references providing additional cGAS inhibitors, cGAS inhibitor scaffolds and motifs, and design considerations include Decout et al., Nat. Rev. Immunol., September 2021; 21 (9): 548-569: Vincent et al., Nat. Commun., 2017, 8:750; Lama et al., Nat. Commun., 2019 May 21; 10 (1): 2261; Obioma et al., U.S. Pat. No. 10,738,056; Hong et al., Journal of Molecular Cell Biology, 2022, 14 (2), njac005; Zhao et al., J. Chem. Inf. Model, 2020, 60, 3265-3276; and Padilla-Salinas et al., J. Org. Chem., 2020, 85, 1579-1600; each of these publications are hereby incorporated by reference herein in their entirety.

In different embodiments the inhibitor is as provided in Table 2 or a pharmaceutically salt thereof.

TABLE 2

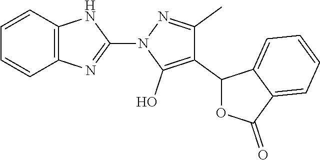 [SMILES: Cc1nn(-c2nc3ccccc3[nH]2)c(O)c1C1OC(=O)c2ccccc21]

RU-354

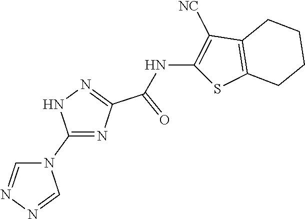 [SMILES: N#Cc1c(NC(=O)c2n[nH]c(-n3cnnc3)n2)sc2c1CCCC2]

Compound S3

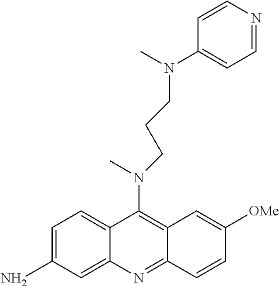 [SMILES: COc1ccc2nc3cc(N)ccc3c(N(C)CCCN(C)c3ccncc3)c2c1]

Compound X6

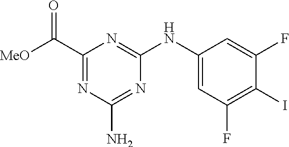 [SMILES: COC(=O)c1nc(N)nc(Nc2cc(F)c(I)c(F)c2)n1]

CU76

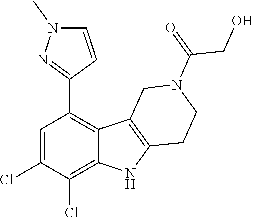 [SMILES: Cn1ccc(-c2cc(Cl)c(Cl)c3[nH]c4c(c23)CN(C(=O)CO)CC4)n1]

G140

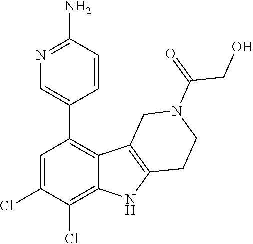 [SMILES: Nc1ccc(-c2cc(Cl)c(Cl)c3[nH]c4c(c23)CN(C(=O)CO)CC4)cn1]

G150

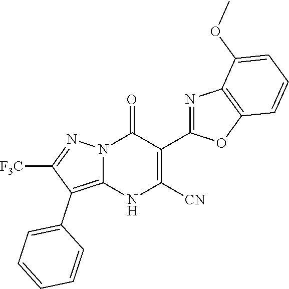 [SMILES: COc1cccc2oc(-c3c(C#N)[nH]c4c(-c5ccccc5)c(C(F)(F)F)nn4c3=O)nc12]

C1089

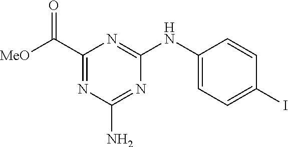 [SMILES: COC(=O)c1nc(N)nc(Nc2ccc(I)cc2)n1]

CU32

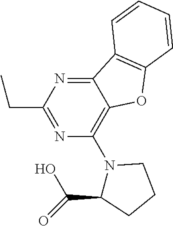 [SMILES: CCc1nc(N2CCC[C@H]2C(=O)O)c2oc3ccccc3c2n1]

Compound 15

In different embodiments the TBK1 inhibitor is either dovitinib,

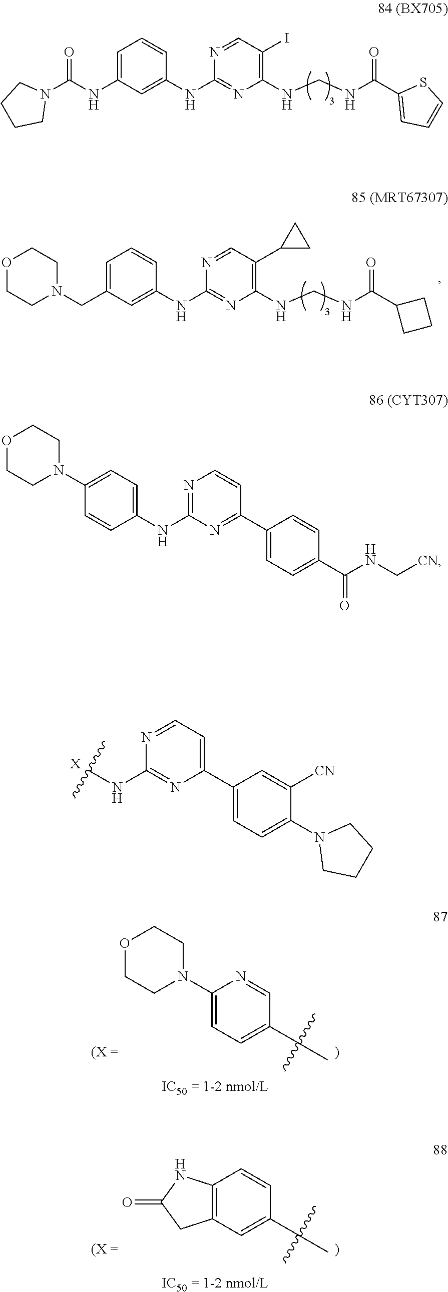

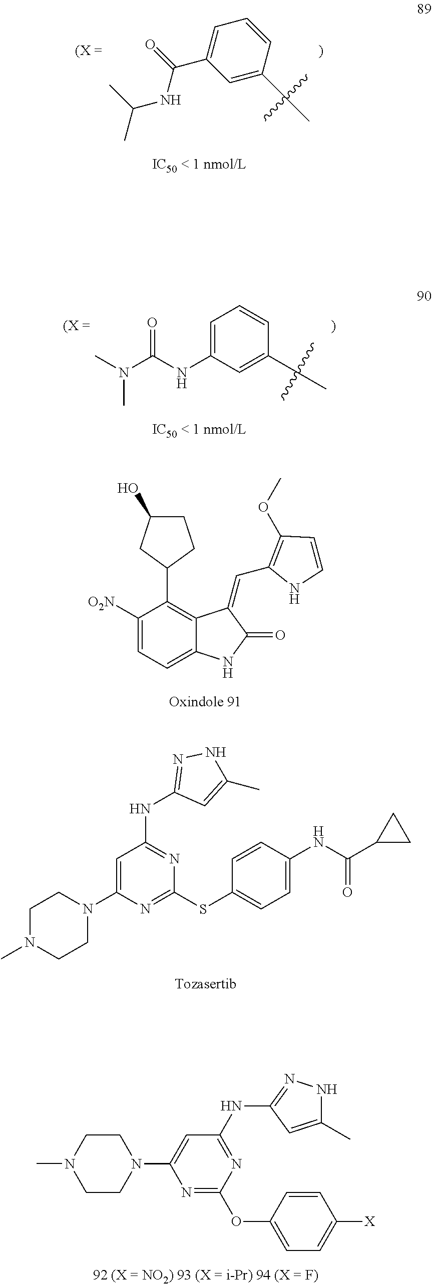

where R is COOH (95) or

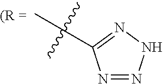

(96)

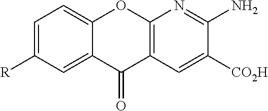

where R is either i-pentyl (97) or c-hexyl (98),

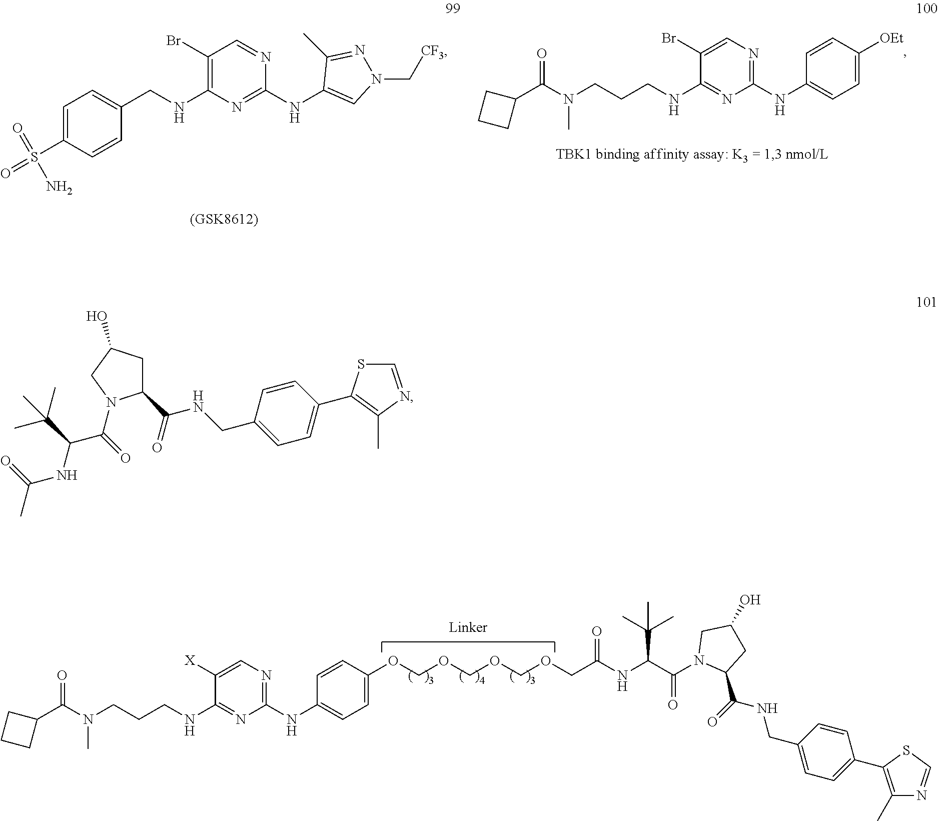

where X is either Br or I,

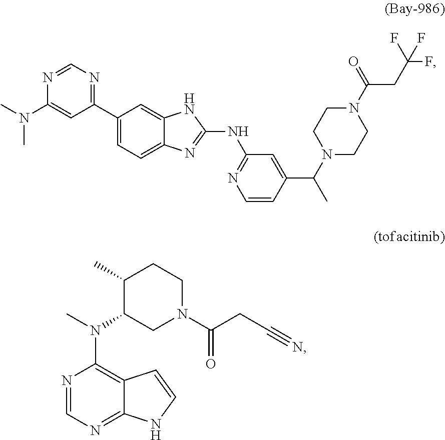 [SMILES: CC(c1ccnc(Nc2nc3ccc(-c4cc(N(C)C)ncn4)cc3[nH]2)c1)N1CCN(C(=O)CC(F)(F)F)CC1 ; C[C@@H]1CCN(C(=O)CC#N)C[C@@H]1N(C)c1ncnc2[nH]ccc12]

or a pharmaceutically acceptable salt thereof.

Additional references providing additional TBK1 inhibitors, TBK1 inhibitor scaffolds and motifs, and design considerations include Thomson et al., Expert Opinion on Therapeutic Patents, 2021, 31:9 785-794; Chekler et al., U.S. Application Patent Publication No. 20210214339; Newton and Stewart, U.S. Pat. No. 11,058,686; Karra et al., International Patent Publication No. WO2019/079373A1; Bigi et al., U.S. Pat. No. 9,994,547; Schulze et al., U.S. Pat. No. 10,894,784; Hassan and Yan, Parmacol. Res., 2016, 111:336-342; Li et al., Int. J. Cancer 2014, 134:1972-1980; Alam et al., International Journal of Biological Macromolecules, 2022, 2027:1022-1037; Perrior et al., U.S. Pat. No. 8,962,609; and Du et al., U.S. Pat. No. 10,316,049; each of these publications are hereby incorporated by reference herein in their entirety.

In certain embodiments the inhibitor is as provided in Table 3 or a pharmaceutically salt thereof.

TABLE 3

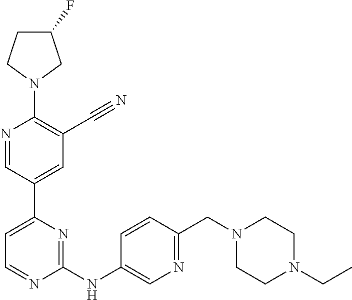 [SMILES: CCN1CCN(Cc2ccc(Nc3nccc(-c4cnc(N5CC[C@H](F)C5)c(C#N)c4)n3)cn2)CC1]

TBK-1

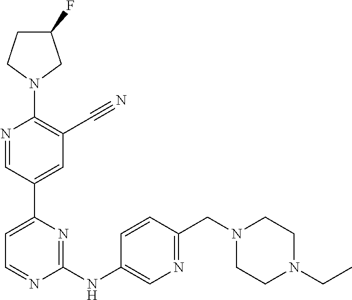 [SMILES: CCN1CCN(Cc2ccc(Nc3nccc(-c4cnc(N5CC[C@@H](F)C5)c(C#N)c4)n3)cn2)CC1]

TBK-2

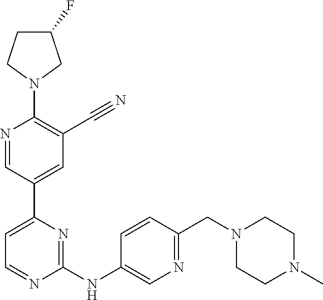 [SMILES: CN1CCN(Cc2ccc(Nc3nccc(-c4cnc(N5CC[C@H](F)C5)c(C#N)c4)n3)cn2)CC1]

TBK-3

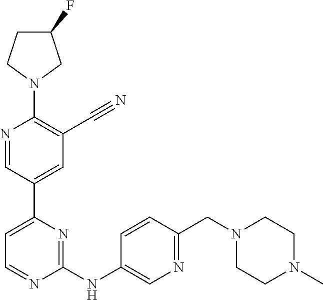 [SMILES: CN1CCN(Cc2ccc(Nc3nccc(-c4cnc(N5CC[C@@H](F)C5)c(C#N)c4)n3)cn2)CC1]

TBK-4

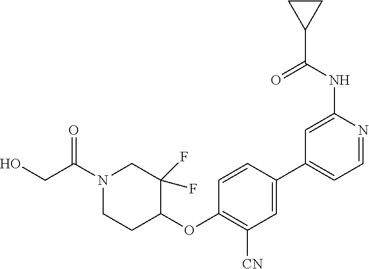 [SMILES: N#Cc1cc(-c2ccnc(NC(=O)C3CC3)c2)ccc1OC1CCN(C(=O)CO)CC1(F)F]

G-56

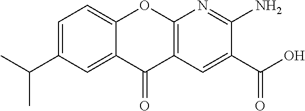 [SMILES: CC(C)c1ccc2oc3nc(N)c(C(=O)O)cc3c(=O)c2c1]

Amlexanox

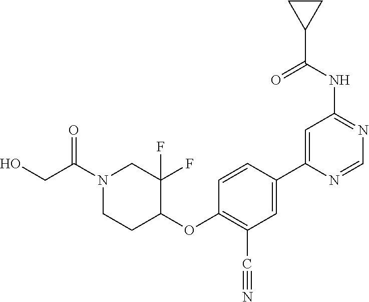 [SMILES: N#Cc1cc(-c2cc(NC(=O)C3CC3)ncn2)ccc1OC1CCN(C(=O)CO)CC1(F)F]

G-55

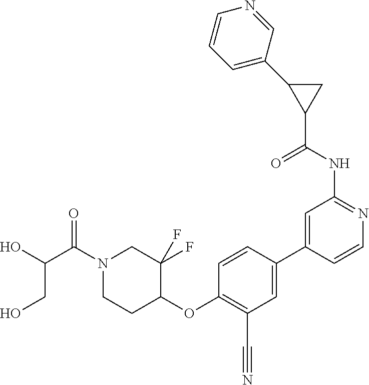 [SMILES: N#Cc1cc(-c2ccnc(NC(=O)C3CC3c3cccnc3)c2)ccc1OC1CCN(C(=O)C(O)CO)CC1(F)F]

G-59

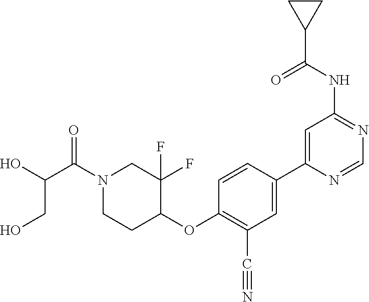 [SMILES: N#Cc1cc(-c2cc(NC(=O)C3CC3)ncn2)ccc1OC1CCN(C(=O)C(O)CO)CC1(F)F]

G-57

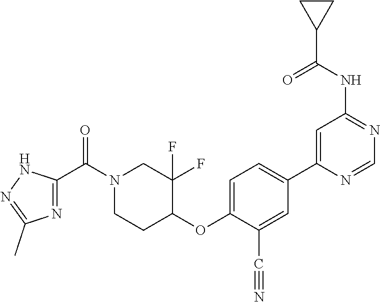 [SMILES: Cc1n[nH]c(C(=O)N2CCC(Oc3ccc(-c4cc(NC(=O)C5CC5)ncn4)cc3C#N)C(F)(F)C2)n1]

G-58

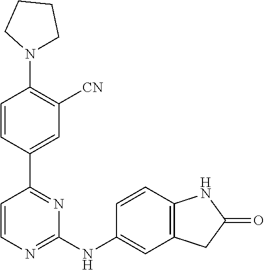 [SMILES: N#Cc1cc(-c2ccnc(Nc3ccc4c(c3)CC(=O)N4)n2)ccc1N1CCCC1]

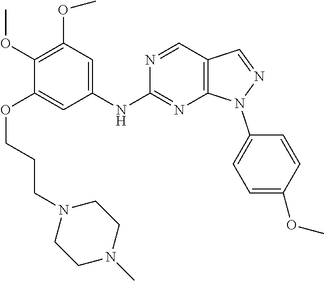 [SMILES: COc1ccc(-n2ncc3cnc(Nc4cc(OC)c(OC)c(OCCCN5CCN(C)CC5)c4)nc32)cc1]

Compound II

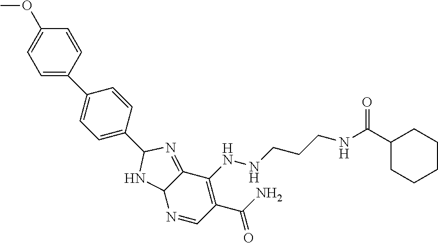 [SMILES: COc1ccc(-c2ccc(C3N=C4C(NNCCCNC(=O)C5CCCCC5)=C(C(N)=O)C=NC4N3)cc2)cc1]

AZ13102909

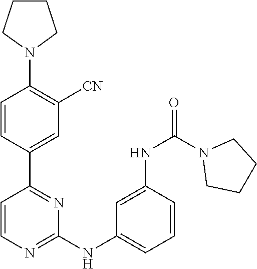 [SMILES: N#Cc1cc(-c2ccnc(Nc3cccc(NC(=O)N4CCCC4)c3)n2)ccc1N1CCCC1]

SR8185

### II.B. Inflammasome Pathway Inhibitor

Inflammasome pathway inhibitors directly affect an inflammasome pathway protein such as Absent in Melanoma-2 (AIM2) protein or can affect an agent impacting the inflammasome pathway. Certain DNA sequences such as the TTAGGG repeat commonly found in mammalian telomeric DNA can bind to AIM2 and suppress innate immune activation. (Kaminski et al., The Journal of Immunology, 2013, 191, 3876.) Such sequences can also inhibit other innate responses such as the cGAS and STING. Examples of AIM2 inhibitors include A151, a synthetic oligonucleotide containing four repeats of the TTAGGG motif, having the following nucleotide sequence, where the bases are joined by phosphorothioate-linkages: 5′-TTAGGGTTAGGGTTAGGGTTAGGG-3′ (SEQ ID NO: 1); and 5′-TTAGGGTTAGGGTTAGGGTTAGGG-3′ (SEQ ID NO: 2), containing phosphodiester linkages. Additional oligonucleotide sequences include other types of modified SEQ ID NO: 2, such those having the same nucleotide sequence, but different modified backbones. Such nucleotide sequences can also be used as cGAS and STING inhibitors and can be incorporated into the DNA or DNA vector.

### III. DNA Vector

DNA vectors comprise a transgene and one or more regulatory elements affecting RNA expression or processing from the transgene. The produced RNA can, for example, be functional or encode for particular protein. Regulatory element may include different elements that, for example, modulate transcription of functional RNA, production of transgene encoded protein and protein processing. Regulatory elements that may be present include an enhancer sequence, intron, post-transcriptional regulatory elements, polyadenylation and termination signal sequences, on-off switches, cell-specific regulators, and internal ribosome entry sites. Depending on the DNA vector, the vector may contain elements in addition to regulatory elements, such as terminal inverted repeats, elements facilitating plasmid replication and selection, and sequences facilitating protein secretion. Multiple transgenes which may be the same type or different; and/or multiple elements which may be of the same type or different may be present.

The DNA vector may further comprise one or more regulatable elements. Such elements provide on/off switches for gene expression. Regulatable elements include tissue-specific and drug-responsive transcription (promoters/enhancers) elements. Examples of regulatable elements include tetracycline inducible elements, druggable ribozymes, druggable toe-hold switches, microRNA responsive genes (e.g., mRNA stability or protein translation), morpholino-responsive mRNAs (e.g., splicing or mRNA stability), suppressor-tRNA regulated genes, genes regulated by alternative splicing, and druggable degrons.

In an embodiment, the DNA vector is used for gene therapy. Gene therapy includes both loss-of-function and gain-of-function genetic defects. The term “loss-of-function” in reference to a genetic defect, refers to a mutation in a gene in which the protein encoded by the gene exhibits either a partial or a full loss of function that is normally associated with the wild-type protein. The term “gain-of-function” in reference to a genetic defect refers to a mutation in a gene in which the protein encoded by the gene acquires a function not normally associated with the wild type protein causes or contributes to a disease or disorder. The gain-of-function mutation can be a deletion, addition, or substitution of a nucleotide or nucleotides in the gene, giving rise to a change in the encoded protein function. In certain embodiments, the gain-of-function mutation changes the function of the mutant protein or causes interactions with other proteins. In certain embodiments, the gain-of-function mutation causes a decrease in or removal of normal wild-type protein, for example, by interaction of the altered, mutant protein with the normal, wild-type protein.

Different types of the DNA vectors may be employed including a minicircle, a nanoplasmid, open linear duplex DNA, closed-ended linear duplex DNA (CELiD/ceDNA/doggybone DNA), single-stranded circular DNA and single-stranded linear DNA.

In an embodiment, the DNA vector balances the particular mammal chosen as a subject, motifs enhancing gene expression; and sequences and motifs that induce immune stimulation in a mammal. Gene expression in a particular mammal can be enhanced, for example, by codon optimization, reduction of CpG and reduction of RNA secondary structure and unstable motifs. Examples of immune stimulating motifs that be reduced include CpG, pyrimidine-rich sequences and palindrome sequences.

In different embodiments, the transgene encodes a viral antigen, a bacterial antigen, a therapeutic protein, a short hair pin RNA (shRNA), a small interfering RNA (siRNA), a microRNA (miRNA), a RNAᵢ, a ribozyme, an antisense RNA, a clustered regularly interspaced short palindromic repeats (CRISPR)/Cas9 construct, a zinc finger nuclease (ZFN), or a transcription activator-like effector nuclease (TALEN).

### III.A. Promoter

Promoters are generally located 5′ of the polynucleotide sequence being expressed and are operatively linked to the polynucleotide sequence. For example, a promoter is operatively linked with a polynucleotide sequence when it is capable of affecting the expression of sequence (e.g., the sequence is under the transcriptional control of the promoter). The promoter binds RNA polymerase and the necessary transcription factors to initiate transcription from the polynucleotide sequence. Promoter sequences define the direction of transcription and which DNA strand will be transcribed.

Encoding sequences can be operatively linked to regulatory sequences in a sense or antisense orientation. In certain embodiments, the promoter is a heterologous promoter. The term “heterologous promoter” refers to a promoter that is not found to be operatively linked to a given encoding sequence in nature.

The promoter sequence can provide proximal and more distal upstream elements. In an embodiment, the promoter comprises an enhancer element. An “enhancer” is a nucleotide sequence that can stimulate promoter activity and can be an innate element of the promoter or a heterologous element inserted to enhance the level or tissue-specificity of a promoter.

The promoter can be derived from different sources or produced from different elements. For example, the promoter can be entirely from a native gene, composed of different elements derived from different naturally occurring promoters, or comprise a synthetic nucleotide sequence.

Different promoters can be selected to direct the expression of a nucleotide sequence in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters are well known in the art. Examples of promoters include the phosphoglycerate kinase (PKG) promoter, CAG (composite of the CMV enhancer the chicken beta actin promoter (CBA) and the rabbit beta globin intron.), NSE (neuronal specific enolase), NeuN promoters, the SV40 early promoter, mouse mammary tumor virus LTR promoter, adenovirus major late promoter (Ad MLP), herpes simplex virus (HSV) promoter, cytomegalovirus (CMV) promoter such as the CMV immediate early promoter region (CMVIE), SFFV promoter, Rous sarcoma virus (RSV) promoter, synthetic promoters, and hybrid promoters. Other promoters can be of human origin or from other species, including mice. Common promoters include the human cytomegalovirus (CMV) immediate early gene promoter, the Rous sarcoma virus long terminal repeat, [beta]-actin, rat insulin promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter. In addition, sequences derived from non-viral genes, such as the murine metallothionein gene, may also be employed. A variety of promoter sequences are commercially available, see e.g., Stratagene (San Diego, CA).

### III.B. Additional Elements

Additional elements that may be present include an intron, an enhancer, a polyadenylation and termination signal, a post translational regulatory element, 5′ and 3′ inverted repeats (ITRs), on-off switches (e.g., rheostats), cell-specific regulators (e.g., micoRNA binding elements), internal ribosome entry sites or other elements that affect expression or stability of the encoded sequence; or protein processing (e.g., secretory signal sequence). An example of the arrangements of different elements is 5′ to 3′ promoter/enhancer, transgene, posttranscription regulatory element, and polyadenylation and termination signal.

Polyadenylation sequences are important for mRNA nuclear transport, translation, and stability. Examples of polyadenylation sequences include HGH, SV40 late, bGHpA, synthetic polyA (SPA), bGH, mutation BGH, and HSV TK. (Wang and Guo 2020, 104, 5673; and Powell et al., Discovery Medicine 2015, 19 (102), 49.)

Viral post-transcriptional regulatory elements are cis-acting elements involved in nuclear export of intronless viral RNA. Examples include hepatitis B virus PRE and woodchuck response element (WRE). (Powell et al., Discovery Medicine 2015, 19 (102), 49.)

The presence of an intron between the promoter and transgene can enhance gene expression and RNA processing. (Powell et al., Discovery Medicine 2015, 19 (102), 49.) Examples of introns include MVM, hCMV intron, intron hEF1 promoter, chimeric intron, modified SV40 intron, and β-globin intron.

If desired, an encoded polypeptide can be expressed with a secretory signal sequence facilitating extracellular secretion of the polypeptide. The term “secretory signal sequence” refers to amino acid sequences functioning to enhance secretion of an operatively linked polypeptide from the cell as compared to the level of secretion seen with the polypeptide lacking the secretory signal sequence. It is not necessary that essentially all or even most of the polypeptide is secreted, as long as the secretion level is enhanced as compared with the native polypeptide. In different embodiments, at least 95%, 97%, 98%, or 99% of the polypeptide is secreted. Generally, secretory signal sequences are cleaved within the endoplasmic reticulum and may be cleaved prior to secretion. It is not necessary that the secretory signal sequence is cleaved as long as secretion of the polypeptide from the cell is enhanced and the polypeptide is functional.

The secretory signal sequence can be derived in whole or in part from the secretory signal of a secreted polypeptide (i.e., from the precursor) and/or can be in whole or in part synthetic. The length of the secretory signal sequence is not critical, and can be for example, from about 10-15 to 50-60 amino acids in length. Known secretory signals from secreted polypeptides can be altered or modified (e.g., by substitution, deletion, truncation, or insertion of amino acids) as long as the resulting secretory signal sequence functions to enhance secretion of an operatively linked polypeptide. The secretory signal sequences can comprise, consist essentially of, or consist of a naturally occurring secretory signal sequence or a modification thereof. Examples of synthetic or artificial secretory signal peptides are provided in Barash et al., Biochem. Biophys. Res. Comm. 294, 835 (2002).

### III.D. Therapeutic Proteins

The DNA vector can deliver a variety of different transgene that can be expressed to provide a protein having a desired activity. Examples of transgenes include those providing a healthy copy of gene in a subject where the gene is defective or a new, a modified gene that can help treat a disease or disorder, or a new gene encoding for protein providing a beneficial effect.

In different embodiments, a transgene encodes GAA (acid alpha-glucosidase) for treatment of Pompe disease; TPP1 (tripeptidyl peptidase-1) for treatment of late infantile neuronal ceroid lipofuscinosis type 2 (CLN2); ATP7B (copper transporting ATPase2) for treatment of Wilson's disease; alpha galactosidase for treatment of Fabry disease; ASS1 (arginosuccinate synthase) for treatment of Citrullinemia Type 1; beta-glucocerebrosidase for treatment of Gaucher disease Type 1; beta-hexosaminidase A for treatment of Tay-Sachs disease; SERPING1 (C1 protease inhibitor or C1 esterase inhibitor) for treatment of hereditary angioedema (HAE), also known as C1 inhibitor deficiency type I and type II); or glucose-6-phosphatase for treatment of glycogen storage disease type I (GSDI).

In different embodiments, the transgene encodes insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor α (TGFα), platelet-derived growth factor (PDGF), insulin growth factors I or II (IGF-I or IGF-II), TGFβ, activins, bone morphogenic protein (BMP), nerve growth factor (NGF), brain-derived neurotrophic factor (BDNF), neurotrophins NT-3 or NT4/5, ciliary neurotrophic factor (CNTF), glial cell line derived neurotrophic factor (GDNF), neurturin, agrin, netrin-1 or netrin-2, hepatocyte growth factor (HGF), ephrins, noggin, sonic hedgehog or tyrosine hydroxylase.

In different embodiments, the transgene encodes thrombopoietin (TPO), an interleukin (IL-1 through IL-36), monocyte chemoattractant protein, leukemia inhibitory factor, granulocyte-macrophage colony stimulating factor, Fas ligand, tumor necrosis factors α or β, interferons α, β, or γ, stem cell factor, flk-2/flt3 ligand, IgG, IgM, IgA, IgD or IgE, chimeric immunoglobulins, an antibody, humanized antibodies, single chain antibodies, T cell receptors, chimeric T cell receptors, single chain T cell receptors, class I or class II MHC molecules. Antibodies and immunoglobulins can, for example, be provided targeting cancer cells or other disease or disorder causing cells.

In different embodiments, the transgene encodes CFTR (cystic fibrosis transmembrane regulator protein), a blood coagulation (clotting) factor (Factor XIII, Factor IX (FIX), Factor VIII (FVIII), Factor X, Factor VII, Factor VIIa, or protein C) a gain of function blood coagulation factor, erythropoietin, LDL receptor, lipoprotein lipase, ornithine transcarbamylase, β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor, epidermal growth factor, nerve growth factor, neurotrophic factor-3 and -4, brain-derived neurotrophic factor, glial derived growth factor, transforming growth factor α and β, a cytokine, α-interferon, β-interferon, interferon-γ, interleukin-2, interleukin-4, interleukin 12, granulocyte-macrophage colony stimulating factor, lymphotoxin, a suicide gene product, herpes simplex virus thymidine kinase, cytosine deaminase, diphtheria toxin, cytochrome P450, deoxycytidine kinase, tumor necrosis factor, a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1, insulin, glucokinase, guanylate cyclase 2D (LCA-GUCY2D), retinal pigment epithelium-specific 65 kDa protein (RPE65), Rab escort protein 1 (choroideremia), LCA 5 (LCA-lebercilin), ornithine ketoacid aminotransferase (gyrate atrophy), retinoschisin 1 (X-linked retinoschisis), X-linked retinitis pigmentosa GTPase (XLRP), MER proto-oncogene tyrosine kinase (MERTK) (autosomal recessive (AR) forms of retinitis pigmentosa (RP)), ABCA4 (Stargardt), ACHM 2, 3 and 4 (achromatopsia), an anti-vascular endothelial growth factor (VEGF) agent polypeptide (e.g., bevacizumab, brolucizumab, ranibizumab, aflibercept), DFNB1 (connexin 26 deafness), USHIC (Usher's syndrome 1C), PKD-1 or PKD-2 (polycystic kidney disease), TPP1 (tripeptidyl peptidase-1), a sulfatase, N-acetylglucosamine-1-phosphate transferase, cathepsin A, GM2-AP, NPC1, VPC2, a sphingolipid activator protein, or one or more donor sequences used as repair templates for genome editing.

In different embodiments, the transgene encodes erythropoietin (EPO) for treatment of anemia; interferon-alpha, interferon-beta, and interferon-gamma for treatment of various immune disorders, viral infections and cancer; an interleukin (IL), including any one of IL-1 through IL-36, and corresponding receptors, for treatment of various inflammatory diseases or immuno-deficiencies; a chemokine, including chemokine (C—X—C motif) ligand 5 (CXCL5) for treatment of immune disorders; granulocyte-colony stimulating factor (G-CSF) for treatment of immune disorders such as Crohn's disease; granulocyte-macrophage colony stimulating factor (GM-CSF) for treatment of various human inflammatory diseases; macrophage colony stimulating factor (M-CSF) for treatment of various human inflammatory diseases;

keratinocyte growth factor (KGF) for treatment of epithelial tissue damage; chemokines such as monocyte chemoattractant protein-1 (MCP-1) for treatment of recurrent miscarriage, HIV-related complications, and insulin resistance; tumor necrosis factor (TNF) and receptors for treatment of various immune disorders; alpha1-antitrypsin for treatment of emphysema or chronic obstructive pulmonary disease (COPD); alpha-L-iduronidase for treatment of mucopolysaccharidosis I (MPS I); ornithine transcarbamoylase (OTC) for treatment of OTC deficiency; phenylalanine hydroxylase (PAH) or phenylalanine ammonia-lyase (PAL) for treatment of phenylketonuria (PKU); lipoprotein lipase for treatment of lipoprotein lipase deficiency; apolipoproteins for treatment of apolipoprotein (Apo) A-I deficiency; low-density lipoprotein receptor (LDL-R) for treatment of familial hypercholesterolemia (FH); albumin for treatment of hypoalbuminemia; lecithin cholesterol acyltransferase (LCAT); carbamoyl synthetase I; argininosuccinate synthetase; argininosuccinate lyase; arginase; fumarylacetoacetate hydrolase; porphobilinogen deaminase; cystathionine beta-synthase for treatment of homocystinuria; branched chain ketoacid decarboxylase; isovaleryl-CoA dehydrogenase; propionyl CoA carboxylase; methylmalonyl-CoA mutase; glutaryl CoA dehydrogenase; insulin; pyruvate carboxylase; hepatic phosphorylase; phosphorylase kinase; glycine decarboxylase; H-protein; T-protein; cystic fibrosis transmembrane regulator (CFTR); ATP-binding cassette, sub-family A (ABC1), member 4 (ABCA4) for the treatment of Stargardt disease; or dystrophin.

In a further embodiment the transgene encodes a protein for treating a disease or disorder selected from the group consisting of: hereditary angioedema, Pompe disease, hemophilia A, hemophilia B, Fabry, wet macular degeneration, Leber hereditary optic neuropathy, and Stargardt disease.

### III.E. Inhibitory Nucleic Acid

The DNA vector can provide a variety of different transgenes encoding for a variety of different inhibitory nucleic acid such as a short hairpin RNA (shRNA), a small interfering RNA (siRNA), a microRNA (miRNA), a RNAᵢ, a ribozyme, and an antisense RNA. In different embodiments, the inhibitory nucleic acid binds to a gene, a transcript of a gene, or a transcript of a gene associated with a polynucleotide repeat disease selected from the group consisting of a huntingtin (HTT) gene, a gene associated with dentatorubropallidoluysian atrophy (atrophin 1, ATNI), androgen receptor on the X chromosome in spinobulbar muscular atrophy, human Ataxin-1, -2, -3, and -7, Cav2.1 P/Q voltage-dependent calcium channel (CACNAIA), TATA-binding protein, Ataxin 8 opposite strand (ATXN8OS), serine/threonine-protein phosphatase 2A 55 kDa regulatory subunit B beta isoform in spinocerebellar ataxia (type 1, 2, 3, 6, 7, 8, 12 17), FMR1 (fragile X mental retardation 1) in fragile X syndrome, FMR1 (fragile X mental retardation 1) in fragile X-associated tremor/ataxia syndrome, FMR1 (fragile X mental retardation 2) or AF4/FMR2 family member 2 in fragile XE mental retardation; myotonin-protein kinase (MT-PK) in myotonic dystrophy; Frataxin in Friedreich's ataxia; a mutant of superoxide dismutase 1 (SOD1) gene in amyotrophic lateral sclerosis; a gene involved in pathogenesis of Parkinson's disease and/or Alzheimer's disease; apolipoprotein B (APOB) and proprotein convertase subtilisin/kexin type 9 (PCSK9), hypercholesterolemia; HIV Tat, human immunodeficiency virus transactivator of transcription gene, in HIV infection; HIV TAR, HIV TAR, human immunodeficiency virus transactivator response element gene, in HIV infection; C—C chemokine receptor (CCR5) in HIV infection; Rous sarcoma virus (RSV) nucleocapsid protein in RSV infection, liver-specific microRNA (miR-122) in hepatitis C virus infection; p53, acute kidney injury or delayed graft function kidney transplant or kidney injury acute renal failure; protein kinase N3 (PKN3) in advance recurrent or metastatic solid malignancies; LMP2, LMP2 also known as proteasome subunit beta-type 9 (PSMB 9), metastatic melanoma; LMP7, also known as proteasome subunit beta-type 8 (PSMB 8), metastatic melanoma; MECL1 also known as proteasome subunit beta-type 10 (PSMB 10), metastatic melanoma; vascular endothelial growth factor (VEGF) in solid tumors; kinesin spindle protein in solid tumors, apoptosis suppressor B-cell CLL/lymphoma (BCL-2) in chronic myeloid leukemia; ribonucleotide reductase M2 (RRM2) in solid tumors; Furin in solid tumors; polo-like kinase 1 (PLK1) in liver tumors, diacylglycerol acyltransferase 1 (DGAT1) in hepatitis C infection, beta-catenin in familial adenomatous polyposis; beta2 adrenergic receptor, glaucoma; RTP801/Redd1 also known as DNA damage-inducible transcript 4 protein, in diabetic macular edema (DME) or age-related macular degeneration; vascular endothelial growth factor receptor I (VEGFR1) in age-related macular degeneration or choroidal neovascularization; caspase 2 in non-arteritic ischaemic optic neuropathy; keratin 6A N17K mutant protein in pachyonychia congenital; influenza A virus genome/gene sequences in influenza infection; severe acute respiratory syndrome (SARS) coronavirus genome/gene sequences in SARS infection; respiratory syncytial virus genome/gene sequences in respiratory syncytial virus infection; Ebola filovirus genome/gene sequence in Ebola infection; hepatitis B and C virus genome/gene sequences in hepatitis B and C infection; herpes simplex virus (HSV) genome/gene sequences in HSV infection; coxsackievirus B3 genome/gene sequences in coxsackievirus B3 infection; silencing of a pathogenic allele of a gene (allele-specific silencing) like torsin A (TORIA) in primary dystonia, pan-class I and HLA-allele specific in transplant; and mutant rhodopsin gene (RHO) in autosomal dominantly inherited retinitis pigmentosa (adRP).

### III.F Gene Editing

The DNA vector can provide a variety of different transgenes encoding for a variety of different gene editing nucleic acid such as ZEN, TALEN, and CRISPR-Cas9. In different embodiments the gene editing nucleic acid edits a subject's DNA to provide a therapeutic protein as provided in Section III.D. supra., or disrupt a gene as provided in Section III.E. supra.

### IV. Immune Cell Modulators

In some cases, administration of the DNA vector can result in an undesirable immune response due to, for example, DNA vector components, the transgene product being recognized as foreign, or an edited gene producing a protein being regarded as foreign. In such cases, if desired, the host immune response can be reduced, for example using an immune cell modulator or immunosuppressant. In some cases, such as cancer treatment, viral treatment and bacterial treatment certain responses may be advantageous.

### IV.A. Phagocyte-Depleting Agent

A “phagocyte-depleting agent” refers to an agent that depletes or destroys phagocytes in a subject and/or interferes with one or more phagocyte functions. Phagocytes, also referred to herein as phagocytic cells, phagocytic immune cells, phagocyte cells, or phagocyte immune cells, include macrophages, monocytes, neutrophils, and dendritic cells. Langerhans cells are dendritic cells found in the skin. Mast cells are found in many tissues including lung or skin and can also act as phagocytes.

A “monocyte and/or macrophage-depleting agent” refers to an agent that depletes or destroys monocytes and/or macrophages in a subject and/or interferes with one or more monocyte and/or macrophage functions. The monocyte and/or macrophage-depleting agents can target monocytes and/or macrophages. Macrophages are mononuclear phagocytes that are differentiated monocytes. In different tissues, macrophages are referred to by different names. Examples of tissue-specific, or resident, macrophages include Kupffer cells in the liver, intestinal macrophages in the gut, microglial cells in the brain, alveolar macrophages in the lung, resident kidney macrophages, skin macrophages, red pulp macrophages in the spleen, and osteoclasts in bone. Examples of monocyte- and/or macrophage-depleting agents include agents targeting phagocytic immune cell markers, e.g., CD115 inhibiting agents such as anti-CD115 antibodies or CD115 small molecule inhibitors; F4/80 inhibiting agents such as anti-F4/80 antibodies or F4/80 small molecule inhibitors; CD68 inhibiting agents such as anti-CD68 antibodies or CD68 small molecule inhibitors; CD11b inhibiting agents such as anti-CD11b antibodies or CD11b small molecule inhibitors; the chemotherapeutic agent Trabectedin; intralipids; empty liposomes; and bisphosphonates including clodronate. In certain embodiments, the monocyte and/or macrophage-depleting agent is not clodronate. In certain embodiments, clodronate and at least one additional monocyte and/or macrophage-depleting agent are used together.

A “neutrophil-depleting agent” refers to an agent that depletes or destroys neutrophils in a subject and/or interferes with one or more neutrophil functions. The neutrophil-depleting agents target neutrophils. Examples of neutrophil-depleting agents include agents targeting phagocytic immune cell markers such Ly6G inhibiting agents, including anti-Ly6G antibodies or Ly6G small molecule inhibitors; CD177 inhibiting agents including anti-CD177 antibodies or CD177 small molecule inhibitors; CD14 inhibiting agents including anti-CD14 antibodies or CD14 small molecule inhibitors; CD15 inhibiting agents including anti-CD15 antibodies or CD15 small molecule inhibitors; CD11b inhibiting agents including anti-CD11b antibodies or CD11b small molecule inhibitors; CD16 inhibiting agent, including anti-CD16 antibodies or CD16 small molecule inhibitors; CD32 inhibiting agents including anti-CD32 antibodies or CD32 small molecule inhibitors; CD33 inhibiting agents including anti-CD33 antibodies or CD33 small molecule inhibitors; CD44 inhibiting agents including anti-CD44 antibodies or CD44 small molecule inhibitors; CD45 inhibiting agents including anti-CD45 antibodies or CD45 small molecule inhibitors; CD66b inhibiting agents including anti-CD66b antibodies or CD66b small molecule inhibitors; CD18, or inhibiting agents including anti-CD18 antibodies or CD18 small molecule inhibitors; CD62L inhibiting agents including anti-CD62L antibodies or CD62L small molecule inhibitors; and Gr-1 inhibiting agents anti-Gr-1 antibodies or Gr-1 small molecule inhibitors.

A “dendritic cell-depleting agent” refers to an agent that depletes or destroys dendritic cells in a subject and/or interferes with one or more dendrite functions. The dendritic cell-depleting agents can target any dendritic cell. Examples of dendritic cell-depleting agents include agents that target phagocytic immune cell markers such as PDCA1 inhibiting agents, including anti-PDCA1 antibodies or PDCA1 small molecule inhibitors; and CD11c inhibiting agents including anti-CD11c antibodies or CD11c small molecule inhibitors.

A “inhibiting agent” refers to any compound capable of down-regulating, decreasing, reducing, suppressing, or inactivating the amount and/or activity of the targeted protein. Inhibiting agents can be proteins, oligo- and polypeptides, nucleic acids, genes, or chemical molecules. Suitable protein inhibitors can be, for example, monoclonal or polyclonal antibodies which bind to the targeted protein; and small molecules.

Examples of CD115 inhibiting agent include CD115 small molecule inhibitors pexidartinib (PLX-3397), BLZ-945, Linifanib (ABT-869), JNJ-28312141 (Johnson & Johnson), JNJ-40346527 (Johnson & Johnson), PLX7486 (Plexxikon), ARRY-382 (Array BioPharma), anti-CD115 antibody such as AFS98 (Invitrogen or BioCell), 12-3A3-1B10 (Invitrogen), 6C7 (Bioss), Cabiralizumab (FPA008), 25949-1-AP (Proteintech), 1G4 (Abnova), 3G12 (Abnova), 604B5 2E11 (Invitrogen), Emactuzumab (RG-7155; Roche), AMG 820 (Amgen), IMC-CS4, and ROS8G11 (Invitrogen). In certain embodiments, the antibody or antigen-binding fragment thereof is AFS98 (e.g., BioCell BE0213 and Oncogene 1995; 11 (12): 2469-2476).

Suitable Ly6G inhibiting agent, including those known to those skilled in the art, in view of the present disclosure can be used. Examples of anti-Ly6G antibodies include A8 (BioCell BP0075-1) and RB6-8C5 (ab25377).

Intralipid and empty liposomes have been shown to interfere with one or more functions of monocytes and/or macrophages. See, e.g., Liu et al., Biochim Biophys Acta. 2013 June; 1830 (6): 3447-53 and Saunders et al., Nano Lett. 2020 Jun. 10; 20 (6): 4264-4269. Pretreatment with intralipid or empty liposomes can effectively saturate monocyte/macrophage cells and prevent phagocytosis of a non-viral therapeutic agent. Examples of intralipids and empty liposomes include 1141-100ML (Sigma Aldrich), 2B6063 (Baxter), and those described in Liu et al., Biochim Biophys Acta. 2013 June; 1830 (6): 3447-53 and Saunders et al., Nano Lett. 2020 Jun. 10; 20 (6): 4264-4269.

Examples of bisphosphonates include clodronate, pamidronate, ibandronate, alendronate, and zoledronate.

Other examples of “phagocyte-depleting agent” include palbociclib (Ibrance®; Pfizer), cromolyn sodium (Nasalcrom®; Bausch & Lomb).

### IV.B. Immunosuppressant

An immunosuppressant is a compound capable of slowing or halting immune system activity in a subject. A variety of different immune responses can be produced including innate immune responses and humoral immune responses. For example, immune responses include a detectable alteration in Toll receptor activation, lymphokine (e.g., cytokine or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen (e.g., antigen) to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion of cells of the immune system and increased processing and presentation of antigen by antigen presenting cells.

Examples of immunosuppressants include a calcineurin inhibitor, such as cyclosporine, ISA (TX) 247, tacrolimus or calcineurin; a target of rapamycin such as sirolimus, everolimus, FK778 or TAFA-93; interleukin-2 α-chain blocker such as basiliximab and daclizumab; inosine monophosphate dehydrogenase inhibitor, such as mycophenolate mofetil; dihydrofolic acid reductase inhibitor such as methotrexate; immunosuppressive antimetabolite such as azathioprine; JAK inhibitors such as Ruxolitinib; cytokine inhibitors such as an anti-cytokine antibody, e.g., Siltuximab; or a steroid.

In certain embodiments, an immunosuppressant is an anti-inflammatory agent. In certain embodiments, an immunosuppressant is a steroid, e.g., a corticosteroid, prednisone, prednisolone, cyclosporine (e.g., cyclosporine A), mycophenolate; a B cell targeting antibody, e.g., rituximab; a proteasome inhibitor, e.g., bortezomib; a mammalian target of rapamycin (mTOR) inhibitor, e.g., rapamycin; a tyrosine kinase inhibitor, e.g., ibrutinib; an inhibitor of B-cell activating factor (BAFF); or an inhibitor of a proliferation-inducing ligand APRIL) or a derivative thereof. In certain embodiments, the immunosuppressive agent is an anti-IL-1β agent (e.g., anti-IL-1β monoclonal antibody canakinumab (Ilaris®)) or an anti-IL-6 agent (e.g., anti-IL-6 antibody sirukumab or anti-IL-6 receptor antibody tocilizumab (Actemra®)), or a combination thereof.

The term “steroid” refers to a chemical substance comprising three cyclohexane rings and a cyclopentane ring. The rings are arranged to form tetracyclic cyclopentaphenanthrene, i.e., gonane. There are different types of steroids such as corticosteroids and glucocorticosteroids.

The term “corticosteroid” refers to a class of steroid hormones produced in the adrenal cortex or produced synthetically. In certain embodiments, the steroid can be a corticosteroid. Corticosteroids are involved in a wide range of physiologic systems such as stress response, immune response and regulation of inflammation, carbohydrate metabolism, protein catabolismblood electrolyte levels, and behavior. Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Non-limiting examples of corticosteroids include, aldosternone, beclomethasone, beclomethasone dipropionate, betametahasone, betametahasone-21-phosphate disodium, betametahasone valerate, budesonide, clobetasol, clobetasol propionate, clobetasone butyrate, clocortolone pivalate, cortisol, cortisteron, cortisone, deflazacort, dexamethasone, dexamethasone acetate, dexamethasone sodium phosphate, diflorasone diacetate, dihydroxycortison, flucinonide, fludrocortisones acetate, flumethasone, flunisolide, flucionolone acetonide, fluticasone furate, fluticasone propionate, halcinonide, halpmetasone, hydrocortisone, hydroconrtisone acetate, hydrocortisone succinate, 16α-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate.

A variety of generic and brand name corticosteroids are available including: cortisone (CORTONE™ ACETATE™, ADRESON™, ALTESONA™, CORTELANT™, CORTISTAB™, CORTISYL™, CORTOGEN™, CORTONE™, SCHEROSON™); dexamethasone-oral (DECADRON ORAL™, DEXAMETH™, DEXONE™, HEXADROL-ORAL™, DEXAMETHASONE™ INTENSOL™, DEXONE 0.5™, DEXONE 0.75™, DEXONE 1.5™, DEXONE 4™); hydrocortisone-oral (CORTEF™, HYDROCORTONE™); hydrocortisone cypionate (CORTEF ORAL SUSPENSION™); methylprednisolone-oral (MEDROL-ORAL™); prednisolone-oral (PRELONE™, DELTA-CORTEF™ PEDIAPRED™, ADNISOLONE™, CORTALONE™, DELTACORTRIL™ DELTASOLONE™, DELTASTAB™, DI-ADRESON F™, ENCORTOLONE™, HYDROCORTANCYL™, MEDISOLONE™, METICORTELONE™, OPREDSONE™, PANAAFCORTELONE™, PRECORTISYL™, PRENISOLONA™, SCHERISOLONA™, SCHERISOLONE™); prednisone (DELTASONE™, LIQUID PRED™, METICORTENT™ ORASONE 1™, ORASONE 5™, ORASONE 10™, ORASONE 20™, ORASONE 50™, PREDNICEN-M™, PREDNISONE INTENSOL™, STERAPRED™, STERAPRED DS™, ADASONE™, CARTANCYL™, COLISONE™, CORDROL™, CORTAN™, DACORTIN™, DECORTIN™, DECORTISYL™, DELCORTIN™, DELLACORT™ DELTADOME™, DELTACORTENE™, DELTISONA™, DIADRESON™, ECONOSONE™, ENCORTON™, FERNISONE™, NISONA™, NOVOPREDNISONE™, PANAFCORT™, PANASOL™, PARACORT™, PARMENISON™, PEHACORT™, PREDELTIN™, PREDNICORT™, PREDNICOT™, PREDNIDIB™, PREDNIMENT™, RECTODELT™, ULTRACORTEN™, WINPRED™); triamcinoloneoral (KENACORT™, ARISTOCORT™, ATOLONE™, SHOLOG A™, TRAMACORT-D™, TRI-MED™, TRIAMCOT™, TRISTOPLEX™, TRYLONE D™, U-TRI-LONE™). In certain embodiments, a corticosteroid can be dexamethasone, prednisone, prednisolone, triamcinolone, clobetasol propionate, betamethasone valerate, betamethasone dipropionate, or mometasone furoate. Methods of synthesizing steroids and corticosteroids are well known in the art and many are also commercially available.

A corticosteroid, e.g., dexamethasone, can be delivered as free dexamethasone, as a separate LNP composition or as part of the same LNP composition as the DNA. Chen et al., Journal of Controlled Release 2018, 286, 46-54, includes a description of LNP providing nucleic acid and dexamethasone linked to fatty acid.

### V. Pharmaceutical Compositions

A pharmaceutical composition contains one or more active component along with a pharmaceutical acceptable carrier. Reference to “pharmaceutically” or “pharmaceutically acceptable” refers to non-toxic molecular entities suitable for administration and/or storage. Pharmaceutical compositions can comprise more than one therapeutically active agent. Examples of pharmaceutically acceptable carriers include a non-toxic (in the amount used) solid, semi-solid or liquid filler, diluent, encapsulating material, or formulation.

The form of the pharmaceutical compositions, the route of administration, the dosage and the regimen depend upon the condition to be treated, such as the severity of the illness, the age, weight, and sex of the patient. Pharmaceutical compositions for the agents described herein can be formulated for topical, oral, intranasal, parenteral, intraocular, intravenous, intramuscular, or subcutaneous administration.

In an embodiment, the pharmaceutical compositions contain a formulation capable of injection into a subject. Examples of injectable formulation components include isotonic, sterile, saline solutions (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition, depending on the case, of sterilized water or physiological saline, permit the constitution of injectable solutions. The doses used for the administration can be adapted as a function of various parameters such as mode of administration, relevant pathology, and duration of treatment.

Other pharmaceutically acceptable forms include tablets or other solids for oral administration, including time release capsules.

A pharmaceutical composition comprising a DNA vector comprising a therapeutic transgene can be administered to a subject at dose suitable for treating a particular disease or disorder. In different embodiments a suitable dosage can be from about 0.01 mg/kg to about 10 mg/kg of vector per kg body weight of a subject, about 0.01 mg/kg to about 0.1 mg/kg of vector per kg body weight of a subject, about 0.1 mg/kg to about 1.0 mg/kg of vector per kg body weight of a subject, and about 1.0 mg/kg to about 10 mg/kg of vector per body weight of a subject.

A small molecule cytosolic DNA-sensing inhibitor or immune cell modulator inhibitor can be administered to a subject at a suitable dose for a particular disease or disorder. In different embodiments a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

An antibody that targets a phagocytic immune cell marker can be administered to a subject at any suitable dose. For example, a suitable dosage can be from about 0.01 mg/kg to about 5 mg/kg body weight of a subject, wherein the dosage is administered in 1 to 10 total injections.

A CD115 inhibitor such as pexidartinib can be administered to a subject at a suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

A bisphosphonate, e.g., clodronate, can be administered to a subject at any suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

A corticosteroid, e.g., dexamethasone, can be administered to a subject at any suitable dose. For example, a suitable dosage can be from about 0.1 mg/kg to about 100 mg/kg body weight of a subject, about 0.1 mg/kg to about 1 mg/kg, about 1.0 mg/kg to about 10 mg/kg, and about 10.0 mg/kg to about 100 mg/kg.

### VI. Administration and Treatment

The different compounds and compositions described herein can be administered to a subject for different purposes including research purposes and to treat a disease or disorder in a mammal. Preferred uses are to treat a disease of disorder in a human.

Reference to “treatment” or “treat” refers to both prophylactic, and therapeutic treatment of a patient having a disease or disorder. Reference to “prophylactic” treatment indicates a decrease in the likelihood of contracting a disease or disorder or decreasing the potential severity of a disease or disorder. Reference to “therapeutic” indicates a clinical meaningful amelioration in at least one symptom or cause associated with a disease or disorder. Thus, treatments include administration to subjects at risk of contracting the disease or disorder, suspected to have contracted the disease or disorder, as well as subjects who are ill or have been diagnosed as suffering from a disease or disorder and includes suppression of clinical relapse.

The terms “ameliorate”, and “amelioration” refer to a detectable or measurable improvement in a disease or disorder symptom or an underlying cellular response. A detectable or measurable improvement includes a subjective or objective decrease, reduction, inhibition, suppression, limit or control in the occurrence, frequency, severity, progression, or duration of the disease or disorder, or complication caused by or associated with the disease or disorder, or an improvement in a symptom or an underlying cause or a consequence of the disease or disorder, or a reversal of the disease or disorder. For Pompe, an effective amount includes an amount that inhibits or reduces glycogen production or accumulation, enhances, or increases glycogen degradation or removal, improves muscle tone and/or muscle strength and/or respiratory function. For HemA or HemB, an effective amount includes an amount that reduces frequency or severity of acute bleeding episodes in a subject and an amount that reduces clotting time as measured by a clotting assay

The terms “effective amount” and “sufficient amount” is that amount required to obtain a desired effect. Treatment can be carried out by administering a therapeutically effective amount of DNA vector to a subject. A therapeutically effective amount can be provided in single or multiple doses to achieve a therapeutic or prophylactic effect.

Other agents can be administered in an “effective amount” or “sufficient amount” to achieve the desired effect. For an example, an effective amount of DNA sensing inhibitor is the amount provided in single or multiple doses that inhibits the activity of the cGAS-STING pathway or the inflammasome pathway, and provides a decrease in one or more activities of the innate immune response. Similarly, an effective amount of immune cell modulators is that amount provided in single or multiple doses that inhibit that provides a detectable reduction in phagocytes and/or phagocyte function, for example as provided in Section IV.A. infra.; and an effective amount of immunosuppressant is that amount provided in single or multiple doses that inhibits an immune system activity, for example as provided in Section IV.B. infra.

An effective amount can be administered alone or can be administered in combination with another composition, treatment, protocol, or therapeutic regimen. The amount can be proportionally increased, for example, based on the need of the subject, type, status and severity of the disease or disorder treated or side effects.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The DNA vector and the cytosolic DNA-sensing inhibitor when separately administered can be provided in any order or at approximately the same time. In certain embodiments, the inhibitor is administered at least 60 minutes, at least 90 minutes, or at least 120 minutes prior to the DNA vector.

In certain embodiments the cytosolic DNA-sensing inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, up to about 4 hours, up to about 5 hours, up to about 6 hours, up to about 7 hours, up to about 8 hours, up to about 9 hours, up to about 10 hours, up to about 12 hours, up to about 1 day, up to about 2 days, up to about 3 days, up to about 4 days, or up to about a week prior to DNA vector administration. Preferably, the cytosolic DNA-sensing inhibitor is administered at about the same time, up to about 5 minutes, up to about 15 minutes, up to about 30 minutes, up to about 45 minutes, up to about 60 minutes, up to about 90 minutes, up to about 2 hours, up to about 3 hours, or about 4 hours prior to DNA vector administration. In certain embodiments the cytosolic DNA-sensing inhibitor is administered about 15 minutes, about 30 minutes, about 45 minutes, about 60 minutes, about 90 minutes, about 120 minutes, or about 1 day after the DNA.

In certain embodiments the cytosolic DNA-sensing inhibitor is administered at about the same time, about 5 minutes, about 10 minutes, about 15 minutes, about 30 minutes, about 45 minutes, about 60 minutes, about 90 minutes, 2 hours, about 2.5 hours, about 3 hours, about 3.5 hours, or about 4 hours prior to DNA vector administration.

The DNA vector and the cytosolic DNA-sensing inhibitor can be provided in the same nanoparticle to release at about the same time, or the nanoparticle can be designed to delay release of one of the components. In an embodiment, the nanoparticle is designed to release the inhibitor prior to the release of the DNA.

Treatment doses of DNA vector can vary and depend upon the type, onset, progression, severity, frequency, duration, or probability of the disease or disorder to which treatment is directed, the clinical endpoint desired, previous or simultaneous treatments, the general health, age, gender, race or immunological competency of the subject and other factors that will be appreciated by the skilled artisan. The dose amount, number, frequency, or duration can be proportionally increased or reduced, as indicated by any adverse side effects, complications or other risk factors of the treatment or therapy and the status of the subject.

The dose to achieve a therapeutic effect, e.g., DNA vector dose in mg per kilogram of body weight (mg/kg), will also vary based on several factors including route of administration, the level of transgene expression required to achieve a therapeutic effect, the specific disease or disorder treated, host immune response to DNA, host immune response transgene expression product, and the stability of the protein, peptide, or nucleic acid expressed. Based on the guidance provided herein, one skilled in the art can determine a suitable DNA vector dose range to treat a patient having a particular disease or disorder.

The overall level of transgene expression can vary depending upon the use of the DNA vector. In different embodiments of gene therapy providing a therapeutic protein, the provided expression or activity is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the corresponding subject protein.

In certain embodiments, a method according to the instant invention can result in reduction of expression or activity of a protein targeted by a therapeutic nucleic acid. In different embodiments reduction of expression or activity of a protein targeted by a therapeutic nucleic acid is at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% of normal expression of the target protein.

Methods and uses of the instant invention include delivery and administration systemically, regionally or locally, for example, by injection or infusion. Delivery of the compositions in vivo can generally be accomplished, for example, by injection using a conventional syringe, although other delivery methods such as convection-enhanced delivery are envisioned (see, e.g., U.S. Pat. No. 5,720,720). For example, compositions can be delivered subcutaneously, epidermally, intradermally, intrathecally, intraorbitally, intramucosally, intraperitoneally (IP), intravenously (IV), intra-pleurally, intraarterially, orally, intrahepatically, via the portal vein, or intramuscularly. Other modes of administration include oral and pulmonary administration, suppositories, and transdermal applications.

### VI.A. Illustrative Disease and Disorder

Diseases and disorders that can be treated include lung disease (e.g., cystic fibrosis), a blood disorder (e.g., anemia), CNS diseases and disorder, epilepsy, a lysosomal storage disease (e.g., aspartylglucosaminuria), Batten disease, late infantile neuronal ceroid lipofuscinosis type 2 (CLN2), cystinosis, Fabry disease, Gaucher disease types I, II, and III, glycogen storage disease II (Pompe disease), GM2-gangliosidosis type I (Tay-Sachs disease), GM2-gangliosidosis type II (Sandhoff disease), mucolipidosis types I (sialidosis type I and II), II (I-cell disease), III (pseudo-Hurler disease) and IV, mucopolysaccharide storage diseases (Hurler disease and variants, Hunter, Sanfilippo Types A,B,C,D, Morquio Types A and B, Maroteaux-Lamy and Sly diseases), Niemann-Pick disease types A/B, C1 and C2, and Schindler disease types I and II), hereditary angioedema (HAE), a copper or iron accumulation disorder (e.g., Wilson's or Menkes disease), lysosomal acid lipase deficiency, a neurological or neurodegenerative disorder, cancer, type 1 or type 2 diabetes, adenosine deaminase deficiency, a metabolic defect (e.g., glycogen storage diseases), and a disease of solid organs (e.g., brain, liver, kidney, heart).

Glycogen storage disease type II, also called Pompe disease can be treated by methods according to the instant invention. Pompe disease is an autosomal recessive disorder caused by mutations in the gene encoding the lysosomal enzyme acid α-glucosidase (GAA), which catalyzes the degradation of glycogen. The resulting enzyme deficiency leads to pathological accumulation of glycogen and lysosomal alterations in body tissues, resulting in cardiac, respiratory, and skeletal muscle dysfunction.

Blood clotting disorders which can be treated include hemophilia A, hemophilia A with inhibitory antibodies, hemophilia B, hemophilia B with inhibitory antibodies, a deficiency in any coagulation Factor: VII, VIII, IX, X, XI, V, XII, II, von Willebrand factor, or a combined FV/FVIII deficiency, thalassemia, vitamin K epoxide reductase C1 deficiency or gamma-carboxylase deficiency.

Other diseases and disorders that can be treated include bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors), or a platelet disorder such as, Bernard Soulier syndrome, Glanzmann thrombasthenia, or storage pool deficiency.

Other diseases and disorders that can be treated include proliferative diseases (e.g., cancers, tumors and dysplasias), Crigler-Najjar and metabolic diseases like metabolic diseases of the liver; Friedreich ataxia; infectious diseases; viral diseases induced for example by hepatitis B or C viruses, HIV, herpes, and retroviruses; genetic diseases such as cystic fibrosis, dystroglycanopathies, myopathies such as Duchenne muscular myopathy or dystrophy, myotubular myopathy, sickle-cell anemia, sickle cell disease, Fanconi's anemia, diabetes, amyotrophic lateral sclerosis (ALS), myotubularin myopathy, motor neuron diseases such as spinal muscular atrophy (SMA), spinobulbar muscular atrophy, or Charcot-Marie-Tooth disease; arthritis; severe combined immunodeficiencies such as RS-SCID, ADA-SCID or X-SCID; Wiskott-Aldrich syndrome; X-linked thrombocytopenia; X-linked congenital neutropenia; chronic granulomatous disease; clotting factor deficiencies; cardiovascular disease such as restenosis, ischemia, dyslipidemia, and homozygous familial hypercholesterolemia; eye or ocular diseases such as retinitis pigmentosa, X-linked retinitis pigmentosa, autosomal dominant retinitis pigmentosa, recessive retinitis pigmentosa, choroideremia, choroidal neovascularization, gyrate atrophy, retinoschisis, X-linked retinoschisis, macular degeneration, diabetic macular edema (DME), diabetic retinopathy associated with DME, wet age-related macular degeneration (wet AMD or wAMD), macular edema following retinal vein occlusion, non-arteritic ischaemic optic neuropathy, Leber congenital amaurosis, Leber hereditary optic neuropathy, achromatopsia, and Stargardt disease; lysosomal storage diseases such as San Filippo syndrome; hyperbilirubinemia such as CN type I or II or Gilbert's syndrome; glycogen storage disease such as GSDI, GSDII (Pompe disease), GSDIII, GSDIV, GSDV, GSDVI, GSDVII, GSDVIII or lethal congenital glycogen storage disease of the heart.

In certain embodiments, the subject has a disease or disorder that affects or originates in the central nervous system (CNS). In certain embodiments, the disease is a neurodegenerative disease. Non-limiting examples of CNS or neurodegenerative disease include Alzheimer's disease, Huntington's disease, ALS, hereditary spastic hemiplegia, primary lateral sclerosis, spinal muscular atrophy, Kennedy's disease, a polyglutamine repeat disease, or Parkinson's disease. In certain embodiments, the disease is a psychiatric disease, an addiction (e.g., to tobacco, alcohol, or drugs), epilepsy, Canavan's disease, or adrenoleukodystrophy. In certain embodiments, the CNS or neurodegenerative disease is a polyglutamine repeat disease such as, spinocerebellar ataxia (SCA1, SCA2, SCA3, SCA6, SCA7, or SCA17).

### VI.B. Administration Examples of Different Diseases and Conditions

Based on the present application a wide variety of different diseases and disorders can treated. Administration for certain disease or disorders is highlighted in the present section. The provided examples, like the other examples in the present application, are for providing different embodiments and illustration purposes.

For Pompe disease, an effective amount is an amount of GAA that inhibits or reduces glycogen production or accumulation, enhances or increases glycogen degradation or removal, reduces lysosomal alterations in tissues of the body of a subject, or improves muscle tone and/or muscle strength and/or respiratory function in a subject. Effective amounts can be determined, for example, by ascertaining the kinetics of GAA uptake by myoblasts from plasma. Myoblasts GAA uptake rates (K uptake) of about 141-147 nM appear to be effective (e.g., Maga et al., J. Biol. Chem. 2012, 8; 288 (3), 1428). In animal models, GAA activity levels in plasma greater than about 1,000 nmol/hr/mL, for example, about 1,000 to about 2,000 nmol/hr/mL have been observed to be therapeutically effective.

For HemA and HemB it is generally expected that a blood coagulation factor concentration greater than 1% of factor concentration found in a normal individual is needed to change a severe disease phenotype to a moderate one. A severe phenotype is characterized by joint damage and life-threatening bleeds. To convert a moderate disease phenotype into a mild one, it is expected that a blood coagulation factor concentration greater than 5% of normal is needed.

FVIII normal level is about 100-200 ng/ml and FIX levels in normal humans is 5000 ng/ml, but levels can be more or less and still considered normal, due to functional clotting as determined, for example, by an activated partial thromboplastin time (aPTT) one-stage clotting assay. Thus, a therapeutic effect can be achieved such that the total amount of FVIII or FIX in the subject/human is greater than 1% of the FVIII or FIX present in normal subjects/humans, e.g., 1% of 100-300 ng/mL.

### VI.C. Immune Response Considerations

Immune cell modulators and cytosolic DNA-sensing inhibitors can be used to reduce the innate immune response mediated by the cGAS-STING and inflammasome pathways, and transgene expression products. cGAS-STING pathway activation, for example, can induce production of interferon, pro-inflammatory cytokines, and proinflammatory chemokines such as IL-6, IFN-γ, IFN-β, CCL4, CCL5 and CXCL10; and cause harmful effect such as inflammation and cell death. Inflammasome pathway activation can produce pro-inflammatory cytokines such as IL-1B, IL-18, and IL-33; and cause harmful effect such as inflammation and pyroptosis.

Administration of the DNA vector should provide for minimal or absent “undesirable immune response”. The undesirable immune response induced by the DNA is distinct from a desirable immune response in the subject induced by an antigen (e.g., bacterial or viral) if encoded by the vector.

In different embodiments, the safe or therapeutically tolerable immune response relates to a level of immune response in a subject upon administration of the DNA; as determined by inflammation, cytokine release, inflammasome activation, and pyroptosis inhibition

In certain embodiments, methods described herein decrease incidence of toxic cytokine release or “cytokine release syndrome” (CRS) or “severe cytokine release syndrome” (sCRS) or “cytokine storm” that can occur in a subject; or pyroptosis.

Decreasing toxic cytokine release or toxic cytokine levels comprises decreasing or inhibiting production of toxic cytokine levels in a subject, or inhibiting or reducing the incidence of cytokine release syndrome or a cytokine storm in a subject. In certain embodiments, the toxic cytokines comprise pro-inflammatory cytokines. In certain embodiments, pro-inflammatory cytokines comprise IL-6, IFN-γ, IL-1β, or TNF-α, or any combination thereof.

In certain embodiments, cytokine release syndrome is characterized by elevated levels of several inflammatory cytokines and adverse physical reactions in a subject such as low blood pressure, high fever, and shivering. In certain embodiments, CRS is characterized by elevated levels of IL-6, IFN-γ, IL-1β, or TNF-α, or any combination thereof.

In certain embodiments, measurement of cytokine levels or concentration, as an indicator of cytokine storm or pyroptosis, can be expressed as fold increase, percent (%) increase, net increase, or rate of change in cytokine levels or concentration. In certain embodiments, absolute cytokine levels or concentrations above a certain level or concentration can be an indication of a subject undergoing or about to experience a cytokine storm. In certain embodiments, absolute cytokine levels or concentration at a certain level or concentration, for example a level or concentration normally found in a control subject not undergoing non-viral gene therapy, can be an indication of a method inhibiting or reducing the incidence of a cytokine storm in a subject undergoing gene therapy.

The term “cytokine level” can encompass a measure of concentration, a measure of fold change, a measure of percent (%) change, or a measure of rate change. Further, the methods for measuring cytokines in blood, saliva, serum, urine, and plasma are well known in the art.

In certain embodiments, IFN-γ levels can be used as a measure of cytokine storm and/or as a common measure of the effectiveness of a treatment for cytokine storms. In certain embodiments, IL-6 levels can be used as a common measure of cytokine storm and/or as a common measure of the effectiveness of a treatment for cytokine storms. Other cytokines can be used as markers of a cytokine storm, for example TNF-α, IB-1α, IL-8, or IL-13.

In certain embodiments, IL-18 is used as a measure of pyroptosis and/or as a measure of the effectiveness of a treatment for pyroptosis.

Levels of cytokines in a subject can be analyzed, measured or determined before and/or after administration of the DNA. Levels of cytokines in a subject can be analyzed or measured multiple times, before and/or after administration of the DNA. Exemplary methods for analyzing and measuring cytokine levels in a biological sample include mesoscale delivery platform (MSD).

Immune cell modulators described in Section IV infra., can be provided prior to, in between or after administration of the DNA vector or cytosolic DNA-sensing inhibitor. In different embodiments, an immune cell modulator is administered between about one minute to about 1 hour, 2 hours, 3 hours, or 4 hours before the DNA vector comprising the transgene and a pharmaceutically acceptable carrier is administered. In different embodiment, the DNA vector is administered between about one minute to about 1 hour, 2 hours, 3 hours, or 4 hours before the immune cell modulator. In different embodiments, the immune cell modulator is administered at least one day before the DNA, optionally no more than 1 year before the DNA vector, such as no more than 52 weeks, 51 weeks, 50 weeks, 49 weeks, 48 weeks, 47 weeks, 46 weeks, 45 weeks, 44 weeks, 43 weeks, 42 weeks, 41 weeks, 40 weeks, 39 weeks, 38 weeks, 37 weeks, 36 weeks, 35 weeks, 34 weeks, 33 weeks, 32 weeks, 31 weeks, 30 weeks, 29 weeks, 28 weeks, 27 weeks, 26 weeks, 25 weeks, 24 weeks, 23 weeks, 22 weeks, 21 weeks, 20 weeks, 19 weeks, 18 weeks, 17 weeks, 16 weeks, 15 weeks, 14 weeks, 13 weeks, 12 weeks, 11 weeks, 10 weeks, 9 weeks, 8 weeks, 7 weeks, 6 weeks, 5 weeks, 4 weeks, 3 weeks, 2 weeks, 1 week, 6 days, 5 days, 4 days, 3 days, or 2 days before the DNA vector is administered; and optionally prior to DNA vector, for example 1 week, 2 weeks, 3 weeks or 4 weeks.

In certain embodiments, the bisphosphonate and/or immune cell modulators is included within nanoparticle containing the DNA e.g., is encapsulated within a LNP, LPNP, a polymer nanoparticle, a protein-based nanoparticle, or a peptide cage.

### VI.D. Combination Treatments

The DNA vectors described herein can be used in combination with other therapies for a particular disease or disorder.

### VI.E. Kits

Further provided herein is a kit providing in separate containers at least: (a) a pharmaceutical composition comprising a nanoparticle comprising a DNA; (b) a cytosolic DNA-sensing inhibitor selected from the group consisting of: a cGAS-STING pathway inhibitor and an inflammasome pathway inhibitor; and (c) optionally an immune cell modulator. In different aspects and embodiments, the DNA and the cytosolic DNA-sensing inhibitor is as described in any of the first, second, third, fourth aspects and any related embodiments; and the immune cell modulator is as described throughout Section IV supra. The amounts of different components can readily be obtained based on the guidance provided herein including throughout Sections V. and VI. supra. The kit may also provide a label with instructions for administration according to the methods described herein

A number of different aspect and embodiments of the instant invention have been described throughout the application. Nevertheless, the skilled artisan without departing from the spirit and scope of the instant invention, can make various changes and modifications of the instant invention to adapt it to various usages and conditions.

### EXAMPLES

Examples are provided below further illustrating different features of the present invention and methodology for practicing the invention. The provided examples do not limit the claimed invention.

### Example 1: In Vitro H-151 Inhibition of DNA-LNP-Induced IRF Activation

THP1-Dual™ cells (InvivoGen) were seeded in 96-well plates at a density of 45,000 cells/well. After overnight incubation, the cells were treated with 25 ng DNA-LNP/well in the presence of 0, 0.2 or 2.1 ng of small molecule STING inhibitor H-151 (InvivoGen), where the H-151 was provided either a) co-encapsulated with the DNA in the LNPs, or b) provided in a soluble form. After 24 hours incubation, activation of the interferon regulatory factor (IRF) pathway (“fold IRF activity”) was determined by measuring luminescence signal (Lucia luciferase) in the cell culture supernatant. The DNA-LNP was made up of (1) DNA plasmid (nanoplasmid expression cassette encoding human coagulation Factor IX (hFIX)) and (2) Lipid 5 (further described in Sabnis et al. and I.A. infra.), 50%; C14-PEG2000, 1.5%; cholesterol, 38.5%; and DSPC, 10%.

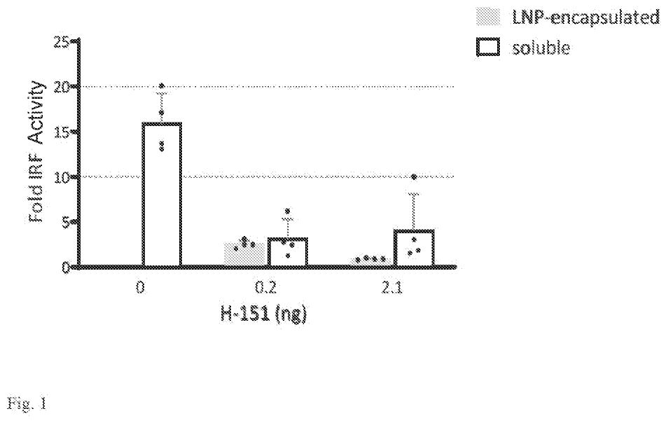

Soluble H-151 had an inhibitory effect on LNP-DNA-induced IRF activation (as demonstrated by cytokine induction), and this effect was more pronounced when the H-151 was encapsulated with the DNA-LNP (FIG. 1).

### Example 2: In Vivo LNP-Encapsulated H-151 Inhibition of DNA-LNP-Induced Cytokine Release

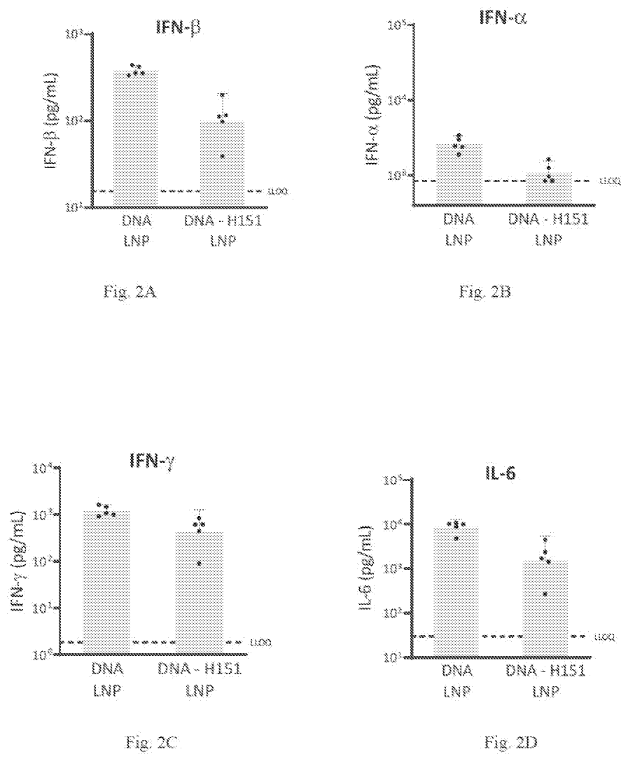

BALB/c mice were injected with 10 μg DNA-LNP or 10 μg DNA-LNP encapsulating H-151. H-151 was provided at a dose of about 1-2 μg/mouse (about 0.04-0.08 mg/kg mouse) Cytokine levels in plasma were measured at 4 hours post injection. FIGS. 2A, 2B, 2C, and 2D illustrate the ability of H-151 encapsulated with the DNA-LNP to inhibit DNA-induced IFN-β (FIG. 2A), IFN-α (FIG. 2B), IFN-γ (FIG. 2C) and IL-6 (FIG. 2D). The DNA-LNP was as described in Example 1, above.

### Example 3: In Vivo RO3150 and GSK690693 Inhibition of DNA-LNP-Induced Cytokine Release

BALB/c mice were pre-treated with dexamethasone, GSK690693, or RO3150 before DNA-LNP administration, and plasma cytokine levels were measured at 4 hours post DNA-LNP treatment. The DNA-LNP was made up of (1) DNA plasmid (nanoplasmid expression cassette encoding hFIX); and (2) GenVoy-ILM™ LNP. GenVoy-ILM™ LNP contains ionizable lipid at 50%; DSPC at 10%; cholesterol 37.5%; and stabilizer (PEG-Lipid) at 2.5% (see Roces et al., Pharmaceutics, 2020 12, 1095). Pretreatment was carried out using 200 μg dexamethasone administered intraperitoneally about one hour prior to DNA-LNP administration; using 312.5 μg GSK69063 administered intraperitoneally about one hour prior to DNA-LNP administration; or using 60 μg RO3015 administered intravenously right before DNA-LNP administration.

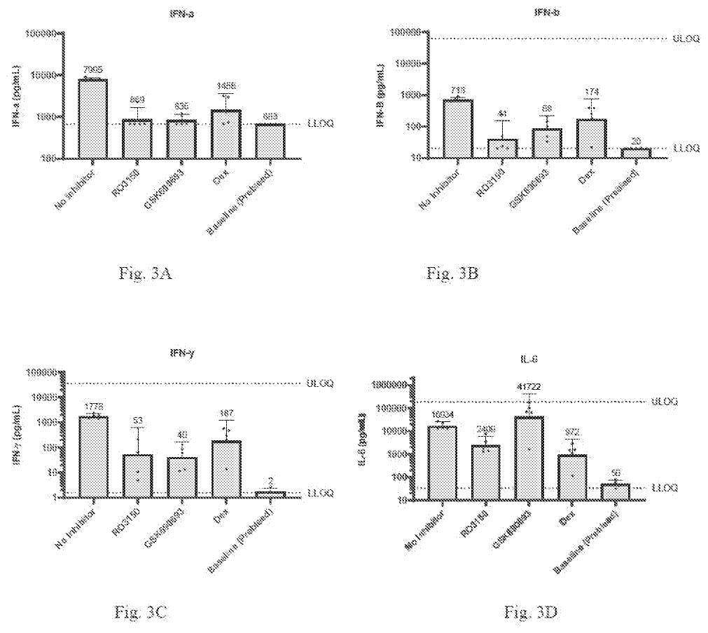

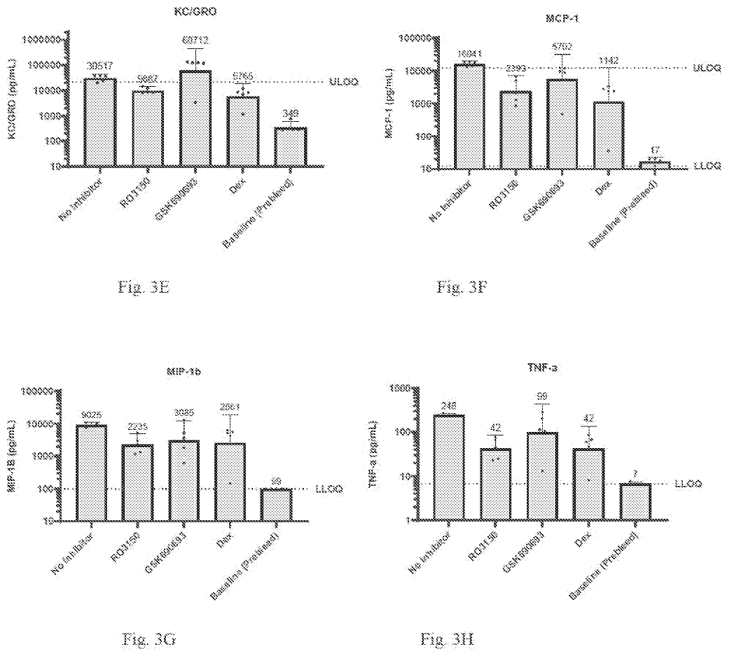

FIGS. 3A, 3B, 3C, 3D, 3E, 3F, 3G, and 3H illustrate the ability of RO3150, GSK690693, and dexamethasone to inhibit cytokine/chemokine levels induced by DNA-LNP. At the employed doses, dexamethasone and RO3150 were broadly active, and GSK690693 reduced most cytokine/chemokine levels except IL-6 and KC/GRO.

### Example 4: Ablation of STING Signaling

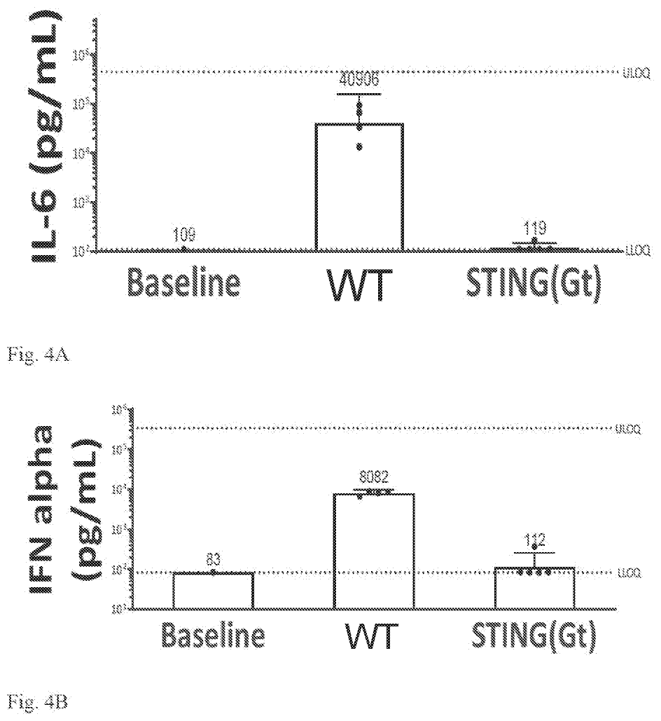

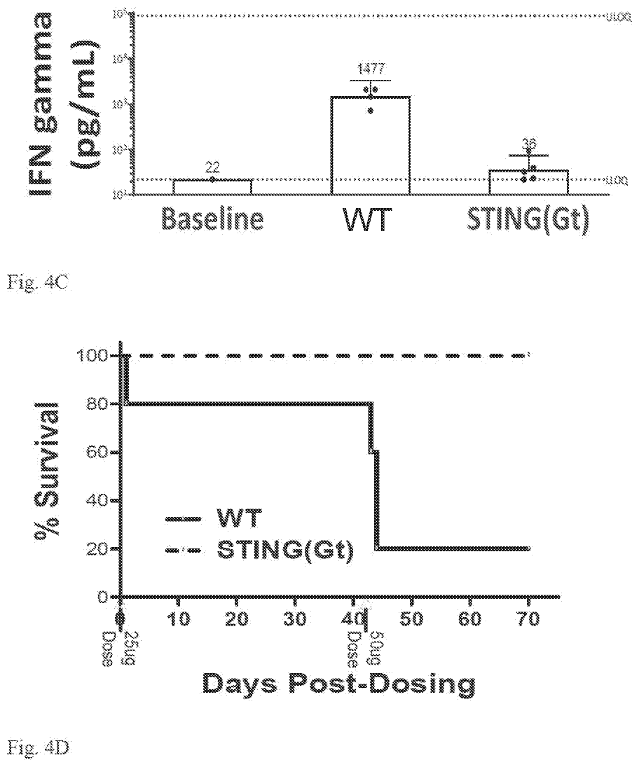

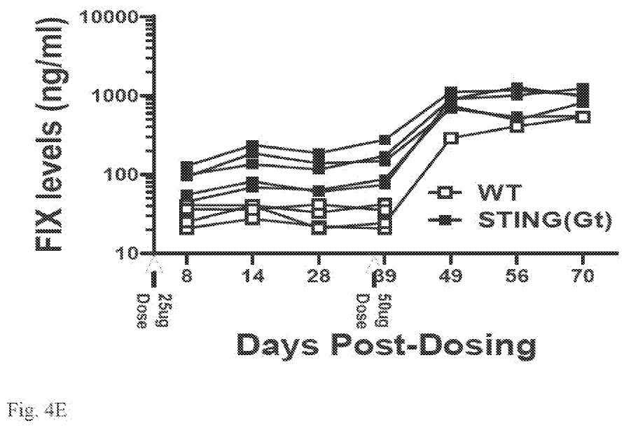

Wild type (WT) mice and mice harboring the Goldenticket (STING (Gt)) missense mutation for the cytosolic double-stranded DNA (dsDNA) sensor STING (n=5 per group) were dosed systemically (bolus in the tail vein) at two timepoints (t=0 days and t=41 days) with DNA-LNPs (0.625 mpk (25 μg/mouse) and 1.25 mpk (50 μg/mouse), respectively). The DNA-LNP comprised a human coagulation factor IX (hFIX)-encoding nanoplasmid DNA encapsulated in an LNP comprising bCKK-E12 lipid (further described in I.A. infra.), 35%; C14-PEG2000, 2.4%; GalNAc PEG C18, 0.1%; cholesterol, 46.5%; and DOPE, 16%. FIGS. 4A, 4B, and 4C show plasma cytokine levels (IL-6, IFN alpha, and IFN gamma, respectively), assayed 4 hours after dosing on day 41, and compared to pooled, pre-dosing plasma levels of WT mice (“Baseline”) (i.e., as measured in WT mice without any DNA-LNP). FIG. 4D shows survival of mice in each group followed out to 70 days. FIG. 4E shows transgenic hFIX protein levels in blood plasma measured by ELISA at various points post-dosing in surviving mice from each group out to day 70. Ablation of STING signaling improves DNA-LNP tolerability, survival, and transgene expression.

Genetic ablation of STING (Goldenticket mice) resulted in a reduction of IL-6 (FIG. 4A), a reduction of IFN alpha (FIG. 4B), a reduction of IFN-gamma (FIG. 4C) and improved survival (FIG. 4D) of mice dosed with a DNA-LNP gene therapy, as compared to WT mice. STING ablation also resulted in increased expression of DNA-LNP gene therapy transgene compared to WT mice (FIG. 4E).

The data support therapeutically targeting STING signaling as an approach to improving tolerability and efficacy of DNA-LNP gene therapies. Potential benefits include reduced sensitivity of immune cells (and possibly also target cells) to an inflammatory response due to the dsDNA payload of the gene therapy treatment.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

I/We claim:

1. A method of intracellular delivery of a DNA to a subject comprising administration of:
a. at least one of a cytosolic DNA-sensing inhibitor selected from the group consisting of a cyclic GMP-AMP synthase-stimulator of interferon genes (cGAS-STING) pathway inhibitor and an inflammasome pathway inhibitor; and
b. a first nanoparticle comprising said DNA wherein step (b) can be performed prior to, concomitantly with, or after step (a).

2. The method of claim 1, wherein if said first nanoparticle is a lipid nanoparticle then at least one of (i) said cytosolic DNA-sensing inhibitor is at least said inflammasome pathway inhibitor; (ii) said lipid nanoparticle does not comprise an endosomolytic agent; (iii) said DNA is circular; (iv) said DNA is not closed-ended DNA; or (v) said cytosolic DNA-sensing inhibitor is provided in a second nanoparticle, wherein said second nanoparticle has the same or different composition as said first nanoparticle.

3. The method of claim 1 or 2, wherein said DNA is a DNA vector comprising a transgene operatively linked to a regulatory element.

4. The method of claim 3, wherein said transgene is operatively linked to a promoter; and said DNA vector comprises 5′ to 3′ said promoter, said transgene, and a polyadenylation and termination signal.

5. The method of claim 4, wherein said promoter is a promoter/enhancer and said vector further comprises a regulatable element.

6. The method of claim 4, wherein said inhibitor is a STING inhibitor.

7. The method of any one of claims 1-5, wherein said cytosolic DNA-sensing inhibitor is a cGAS-STING pathway inhibitor.

8. The method of claim 7, wherein said cGAS-STING pathway inhibitor is a cGAS inhibitor.

9. The method of claim 7, wherein said cGAS-STING pathway inhibitor is a STING inhibitor.

10. The method of claim 7, wherein said cGAS-STING pathway inhibitor is a TBK1 inhibitor.

11. The method of claim 7, wherein said cGAS-STING pathway inhibitor is (a) selected from the group consisting of H-151, GSK-690693, RU-521, RO-3150, CYT387 and GSK8612, or a pharmaceutically acceptable salt thereof; or (b) is a compound of Table 1, Table 2, or Table 3, or a pharmaceutically acceptable salt thereof.

12. The method of any one of claims 1 to 5, wherein said cytosolic DNA-sensing inhibitor is an inflammasome pathway inhibitor.

13. The method of claim 12, wherein said inflammasome pathway inhibitor is an AIM2 inhibitor.

14. The method of claim 13, wherein said inflammasome pathway inhibitor is a polynucleotide having the sequence of SEQ ID NO: 1 or SEQ ID NO: 2.

15. The method of any one of claims 3-14, wherein said transgene encodes a viral antigen, a bacterial antigen, a therapeutic protein, a short hair pin RNA (shRNA), a small interfering RNA (siRNA), a microRNA (miRNA), an RNAᵢ, a ribozyme, an antisense RNA, a clustered regularly interspaced short palindromic repeats (CRISPR)/Cas9 construct, a zinc finger nuclease (ZFN), or a transcription activator-like effector nuclease (TALEN).

16. The method of any one of claims 1-15, wherein said DNA is circular DNA.

17. The method of any one of claims 1-16, wherein said cytosolic DNA-sensing inhibitor is provided in a second nanoparticle, wherein said second nanoparticle has substantially the same composition as said first nanoparticle.

18. The method of any of claims 1-16, where said DNA vector and said cytosolic DNA-sensing inhibitor are provided together in said first nanoparticle.

19. The method of any one of claims 1-18, wherein said first nanoparticle is a lipid nanoparticle or a lipid polymer nanoparticle.

20. The method of claim 19, wherein said first nanoparticle is configured to release said cytosolic DNA-sensing inhibitor prior to the release of said DNA or said DNA vector.

21. The method of any one of claims 1-19, wherein said cytosolic DNA-sensing inhibitor is administered at about the same time up to about 4 hours prior to administration of said DNA or said DNA vector.

22. The method of any one of claims 1-21, wherein both an inflammasome inhibitor and a cGAS-STING inhibitor are administered.

23. The method of any one of claims 1-22, wherein said DNA substantially comprises double-stranded DNA and said DNA vector substantially comprises double-stranded DNA.

24. The method of any one of claims 3-23, wherein said subject is a human patient, and said method provides a therapeutically effective amount of said transgene.

25. A nanoparticle composition comprising
a. a DNA; and
b. at least one cytosolic DNA-sensing inhibitor selected from the group consisting of a cyclic GMP-AMP synthase (cGAS)-STING pathway inhibitor and an inflammasome pathway inhibitor.

26. The composition of claim 25, wherein said DNA is a DNA vector comprising a transgene operatively linked to a regulatory element.

27. The composition of claim 26, wherein said DNA vector comprises 5′ to 3′ a promoter, said transgene, and a polyadenylation and termination signal.

28. The composition of claim 27, wherein said promoter is a promoter/enhancer and said vector further comprises a regulatable element.

29. The composition of any one of claims 25 to 28, wherein said inhibitor is a cGAS-STING pathway inhibitor.

30. The composition of claim 29, wherein said cGAS-STING pathway inhibitor is a cGAS inhibitor.

31. The composition of claim 29, wherein said cGAS-STING pathway inhibitor is a STING inhibitor.

32. The composition of claim 29, wherein said cGAS-STING pathway inhibitor is a TBK1 inhibitor.

33. The composition of claim 29, wherein said cGAS-STING pathway inhibitor is (a) selected from the group consisting of H-151, GSK-690693, RU-521, RO-3150, CYT387 and GSK8612, or a pharmaceutically acceptable salt thereof; or (b) is a compound of Table 1, Table 2, or Table 3, or a pharmaceutically acceptable salt thereof.

34. The composition of any one of claims 25 to 28, wherein said cytosolic DNA-sensing inhibitor is an inflammasome pathway inhibitor.

35. The composition of claim 34, wherein said inflammasome pathway inhibitor is an AIM2 inhibitor.

36. The composition of claim 35, wherein said inflammasome pathway inhibitor is a polynucleotide having the sequence of SEQ ID NO: 1 or SEQ ID NO: 2.

37. The composition of any one of claims 25 to 36, wherein said transgene encodes a viral antigen, a bacterial antigen, a therapeutic protein, a short hair pin RNA (shRNA), a small interfering RNA (siRNA), a microRNA (miRNA), a RNAᵢ, a ribozyme, an antisense RNA, a clustered regularly interspaced short palindromic repeats (CRISPR)/Cas9 construct, a zinc finger nuclease (ZFN), or a transcription activator-like effector nuclease (TALEN).

38. The composition of any one of claims 25 to 37, wherein said DNA vector is circular DNA.

39. The composition of any one of claims 25 to 38, wherein said nanoparticle is a lipid nanoparticle or a lipid polymer nanoparticle.

40. The composition of claim 39, wherein said nanoparticle is a lipid polymer nanoparticle configured to release said cytosolic DNA-sensing inhibitor prior to said DNA vector.

41. The composition of any one of claims 25 to 40, wherein said nanoparticle comprises both an inflammasome inhibitor and a cGAS-STING inhibitor.

42. The composition of any one of claims 25 to 41, wherein said DNA substantially comprises double-stranded DNA and said DNA vector substantially comprises double-stranded DNA.

43. A pharmaceutical composition comprising the nanoparticle composition of any one of claims 25-42 and a pharmaceutically acceptable carrier.

44. The pharmaceutical composition of claim 43, wherein said composition is for use in the methods of any one of claims 1-24.

45. A pharmaceutical composition for use in medicine, preferably gene therapy, comprising the first nanoparticle of any one of claims 1-24, for us in the methods of any one of claims 1-24.


SEQUENCE LISTING (2 sequences)

SEQ ID NO: 1 - DNA, 24 bp, synthetic construct
  source 1..24: mol_type = other DNA
  modified_base 1..24: mod_base = OTHER; note = phosphorothioate-linkages
ttagggttagggttagggttaggg

SEQ ID NO: 2 - DNA, 24 bp, synthetic construct
  source 1..24: mol_type = other DNA
ttagggttagggttagggttaggg